(19) United States
(12) Patent Application Publication
(10) Pub. No.: US 2013/0317847 A1
(43) Pub. Date: Nov. 28, 2013

(54) Medical Care Support System

(71) Applicants: Shuntaro Yui, Tokyo (JP); Kumiko Seto, Tokyo (JP); Masayuki Ohta, Tokyo (JP); Satoshi Mitsuyama, Tokyo (JP)

(72) Inventors: Shuntaro Yui, Tokyo (JP); Kumiko Seto, Tokyo (JP); Masayuki Ohta, Tokyo (JP); Satoshi Mitsuyama, Tokyo (JP)

(73) Assignee: HITACHI, LTD., Chiyoda-ku, Tokyo (JP)

(21) Appl. No.: 13/978,084

(22) PCT Filed: Dec. 8, 2011
(86) PCT No.: PCT/JP2011/078491

§ 371 Date: Aug. 9, 2013

(30) Foreign Application Priority Data
Jan. 4, 2011 (JP) 2011-000018

(51) Int. Cl. G06Q 10/06; G06Q 50/22

(52) U.S. Cl. CPC G06Q 10/0633; G06Q 50/22
USPC 705/2

(57) ABSTRACT

The present invention is a system for supporting an evaluation of workflow step items on the basis of a workflow by classifying workflow steps so as to have a common medical-related work by using a medical information database in which a workflow, which is a flow of the medical-related work, and information on respective workflow steps are stored in association with medical information, creating workflow step items having a parent and child identifier identified between a parent department as a client and a child department as a requested party and the medical information, calculating a workflow step evaluation value, which is an evaluation value of the workflow step item on the basis of the clinical indicator of the workflow, and displaying the same on a screen.

### TECHNICAL FIELD

The present invention relates to a medical care support system configured to treat laboratory data and image data of patients, and remarks of medical practitioners, and to a system configured to support analyses of a medical care process.

### BACKGROUND ART

In a social situation in which improvement of quality and efficiency of medical treatment are promoted, it is important to analyze data in which medical practitioners' knowledge or the like is accumulated and improve a medical care process performed thus far. In the analysis of the medical care process, a clinical path (also referred to as critical path, path) indicating a standard medical care plan is given importance.

PTL 1 (Related Art 1) discloses a technology relating to a system configured to compare a provisional path in which a therapeutic practice set in medical performance data is set as a programmed therapeutic practice with a therapeutic practice acquired on the basis of a patient identifier and register the provisional path whose degree of coincidence exceeds a predetermined reference as a real clinical path.

PTL 2 (Related Art 2) discloses a technology relating to a method of comparing the price, the quality, and the usage of procedure among suppliers providing different medical services for the purpose of determining whether or not a given medical act is important.

### CITED REFERENCE

### Patent Literature

PTL 1: JP-A-2007-265080
PTL 2: JP-T-5-507567

### Non Patent Literature

NPL 1: Measure the quality of Medical Treatment Vol.'(Published by INTER MEDICA Col, Ltd.)

### SUMMARY OF INVENTION

### Technical Problem

A medical-related work for a patient is to be performed along a workflow, which is a flow of a series of medical-related works performed by respective medical practitioners, and reuse of knowledge of the medical practitioner is desired to be performed on the basis of the workflow for enhancement and improvement of the quality of medical care. Therefore, a medical care process analysis from a viewpoint of the workflow is effective.

Since the system disclosed in PTL 1 is a system configured to register medical acts executed in many cases as a clinical path, there is a problem that the medical acts executed in many cases cannot necessarily be said to be an adequate medical act. The method disclosed in PTL 2 described above is a method of comparing the price of the medical service with the prices of a plurality of the medical services adjusted in clinical complexity, and has a problem that the medical-related works cannot be analyzed and evaluated individually.

In this manner, in the technologies disclosed in the related art, since medical information considering a workflow which is a series of medical-related works of the respective medical practitioners is not accumulated, adequacy of the medical knowledge considering the workflow cannot be evaluated. In addition, since the information for reusing the medical information on the basis of the workflow is not accumulated, adequacy of the medical knowledge considering reuse of the medical information or a modification of the medical care process performed at that time cannot be evaluated.

### Solution to Problem

In order to solve the above-described problem, there is provided a medical care support system including: a medical information database in which workflow information configured to identify a workflow as a flow of a series of medical care of a medical-related work, a workflow step item as information configured to identify a workflow step as a unit of medical-related work which constitutes the workflow, a disease identifier configured to identify a disease, a clinical indictor as an indicator of a quantitative evaluation of the quality of medical care are stored in association with each other; a disease name input accepting unit configured to accept an input of a first disease identifier; a workflow information extracting unit configured to extract a first workflow group including a plurality of items of workflow information associated with the first disease identifier whose input is accepted from the medical information database; a workflow clinical evaluation value calculating unit configured to acquire the clinical indicators associated with the respective items of workflow information of the first workflow group respectively from the medical information database and calculate workflow clinical evaluation values, which are clinical evaluation values of the workflow with respect to the respective items of workflow information of the first workflow group on the basis of the acquired clinical indicators; a workflow step evaluation value calculating unit configured to extract a first workflow step item included in the first workflow group from the medical information database respectively and calculate a workflow step evaluation value, which is an evaluation value of the first workflow step item on the basis of the first workflow group and a plurality of the calculated workflow clinical evaluation values; and a screen output unit configured to output the first workflow step item and the calculated workflow step evaluation values to a screen.

### Advantageous Effects of Invention

The evaluation of respective medical acts considering the workflow step, which is a flow of a series of medical-related work of the respective medical practitioners, may be supported. In addition, with workflow step classifying means, classification of the workflow step focusing on the common medical-related work is enabled, so that a comprehensive analysis which absorbs difference in content of processing to some extent is achieved.

### BRIEF DESCRIPTION OF THE DRAWINGS

FIG. 1A illustrates an example of a configuration when a medical care support system is installed in a hospital.

FIG. 1B illustrates an example of another configuration of the medical care support system is installed in a hospital.

FIG. 2A illustrates an example of a structure of a workflow table which constitutes a medical information database.

FIG. 2B illustrates an example of a structure of a workflow step information table which constitutes the medical information database.

FIG. 3A illustrates an example of a structure of a medical information table which constitutes the medical information database.

FIG. 3B illustrates an example of a structure of an evidence data table which constitutes the medical information database.

FIG. 3C illustrates an example of a structure of a processing archival record table which constitutes the medical information database.

FIG. 3D illustrates an example of a structure of an input data table which constitutes the medical information database.

FIG. 4A illustrates an example of a laboratory data table which constitutes a laboratory information database.

FIG. 4B illustrates an example of a test item master table which constitutes the laboratory information database.

FIG. 4C illustrates an example of a measured value table which constitutes the laboratory information database.

FIG. 4D illustrates an example of a measured item master table which constitutes the laboratory information database.

FIG. 4E illustrates an example of an image table which constitutes the laboratory information database.

FIG. 5 illustrates an example of a flowchart which extracts workflow steps from a workflow, and supports the medical care by using medical information and a judgment sentence.

FIG. 6 illustrates an example of a flowchart which supports the medical care by performing data processing on evidence data.

FIG. 7A illustrates an example of a screen before a reference of a test graph and an input of a judgment sentence on the basis of the workflow.

FIG. 7B illustrates an example of a screen when referring to the test graph.

FIG. 7C illustrates an example of a screen when inputting the judgment sentence.

FIG. 8A illustrates an example of a screen before a reference of image data and an input of a judgment sentence on the basis of the workflow.

FIG. 8B illustrates an example of a screen when referring to the image data.

FIG. 8C illustrates an example of a screen when referring to the image data and inputting the judgment sentence on the basis of the workflow.

FIG. 9 illustrates a first example of a configuration of process analyzing means 160.

FIG. 10 illustrates is a first example of a flowchart showing an outline of an operation of the process analyzing means 160.

FIG. 11 is a first example of the flowchart illustrating an operation of workflow step classifying means 1603.

FIG. 12A illustrates an example of a clinical indicator table of the present invention.

FIG. 12B illustrates an example of a workflow step pattern table.

FIG. 12C illustrates an example of an evidence pattern table.

FIG. 12D illustrates an example of a processing archival record pattern table.

FIG. 13 illustrates a first example of a screen of the process analyzing means 160.

FIG. 14 illustrates a second example of the screen of the process analyzing means 160.

FIG. 15 illustrates a third example of the screen of the process analyzing means 160.

FIG. 16 illustrates a fourth example of the screen of the process analyzing means 160.

FIG. 17 illustrates a fifth example of the screen of the process analyzing means 160.

FIG. 18 illustrates a second example of the flowchart showing the outline of the operation of the process analyzing means 160.

FIG. 19 illustrates a third example of the flowchart showing the outline of the operation of the process analyzing means 160.

FIG. 20 is a second example of the flowchart of the operation of the workflow step classifying means 1603.

FIG. 21 illustrates the detailed second example of the flowchart of the operation of the workflow step classifying means 1603.

FIG. 22A illustrates an example of a programmed department of execution classification table in which the programmed department of execution relating to a workflow step master of the present invention is hierarchized.

FIG. 22B illustrates an example of a workflow step master table.

FIG. 22C illustrates an example of an evidence master table.

FIG. 22D illustrates an example of a processing archival record master table.

FIG. 23 illustrates a second example of a configuration drawing of the process analyzing means 160.

FIG. 24 illustrates a fourth example the flowchart of the outline of the operation of the process analyzing means 160.

FIG. 25 illustrates a sixth example of the screen of the process analyzing means 160.

FIG. 26 illustrates a seventh example of the screen of the process analyzing means 160.

FIG. 27 illustrates an eighth example of the screen of the process analyzing means 160.

FIG. 28 illustrates a fifth example the flowchart of the outline of the operation of the process analyzing means 160.

FIG. 29 illustrates a sixth example of the flowchart of the outline of the operation of the process analyzing means 160.

FIG. 30 illustrates an example of a flowchart which calculates of a workflow step reproduction influence rate.

FIG. 31 illustrates a ninth example of the screen of the process analyzing means 160.

FIG. 32 illustrates a tenth example of the screen of the process analyzing means 160.

FIG. 33 illustrates an eleventh example of the screen of the process analyzing means 160.

### DESCRIPTION OF EMBODIMENTS

<Outline of Respective Contents of Description Given Below>

The present invention performs an evaluation of workflows and workflow steps by using information accumulated in a medical information database. As an order of explaining Description of Embodiments, respective items of information accumulated in the medical information database and a structure of the database used in the present invention will be described first. Subsequently, how this system accumulates the respective items of information in the medical information database will be described. Subsequently, an evaluation of the workflows and the workflow steps using the medical information database will be described.

<Description of Definition of Workflow>

The term “workflow” refers to a flow of a series of medical-related works performed for a patient by a medical practitioner, and the term “workflow information” refers to information on identification of the workflow. The term “workflow step” is a unit of the medical-related work constituting the workflow, and the term “workflow step information” includes information for identifying the workflow step and information on either at least a client of the medical-related work or a requested party.

The term “request of the medical-related work” refers to ordering of the medical-related work to respective health-care units, for example, a test department, or an image diagnosing department. The term “medical practitioner” includes not only doctors in charge, laboratory personnel, radiology technicians, and doctors interpreting radiogram, but also clinical nurses and personnel in charge of medical business accountants. The “start” and “termination” of the workflow is decided by a primary doctor among clinicians, for example. The workflow includes a flow of a series of medical cares such as diagnosis, therapy, a wait-and-see approach from one disease to another, a flow of medical care during a hospital stay (from the first day to the last day of the hospital stay), and a flow determined by a medical care guideline.

The workflow step includes, for example, respective works in medical care stages (diagnosis, therapy, wait-and-see approach) of a patient, or a diagnostic work given by the primary doctor, works such as medication performed by the clinical nurses, an ordering work unit interchanged between a health-care unit such as a diagnosis and treatment department and a department of radiology. The workflow step information may also be edited by the medical practitioner or the like in the respective workflow steps by being newly created or deleted. It may also be based on a predetermined medical care guideline. The workflow information includes a patient identifier for identifying the patient relating to the corresponding workflow.

<Description of System Configuration>

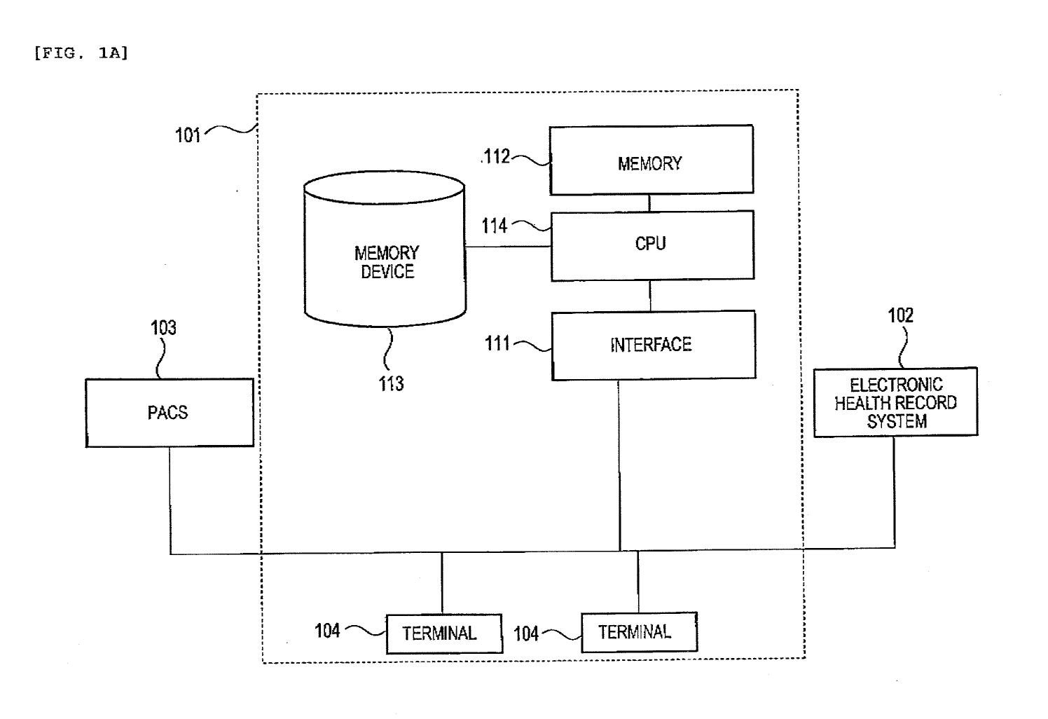

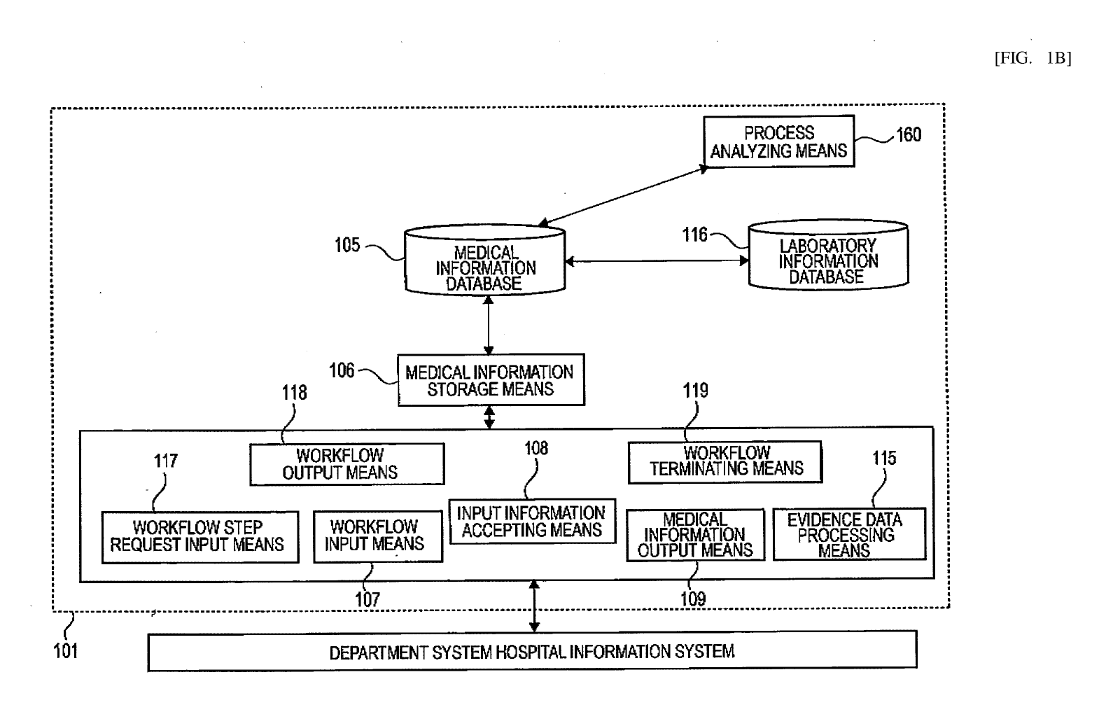

FIG. 1A illustrates an example of a form in which a medical care support system 101 is installed in a hospital. In the medical care support system 101, terminals 104, an interface 111, a memory 112, a memory device 113 such as a hard disk drive are connected to a CPU 114. An operation of the medical care support system is performed by reading a program stored in the memory 112 upon reception of an input via the terminal 104 by the interface 111, and outputting the same to the terminal 104 by using the CPU 114 via the interface 111 or the like. The medical care support system 101 illustrated in FIG. 1A is installed by being connected to an electronic health record system 102 and a PACS 103 or the like via a network. The respective terminals 104 receive manipulated inputs by medical practitioners such as doctors, clinical nurses, laboratory personnel, and doctors interpreting radiogram. The electronic health record system 102 and the PACS 103 may be shared with the medical care support system via the respective terminals 104. There is a case where respective medical care archival record, laboratory data, image data are accumulated in the electronic health record system 102 and the PACS 103. In such a case, the memory device 113 of the medical care support system may have information indicating a link to the database thereof or may have copies of the data stored in the database thereof. As illustrated in FIG. 1B which will be described later, a laboratory information database 116 which is a database owned by the electronic health record system 102 and the PACS 103 may be included in the medical care support system. Examples of the interface 111 include a type provided with a display.

FIG. 1B illustrates an example of a configuration of the medical care support system of the present invention. There is a medical information database 105 in which workflow information and workflow step information and medical information, which is information on the medical-related work are stored so as to be in association with each other, and medical information storage means 106 configured to store such information is connected to the medical information database 105. The medical information includes information input by the medical practitioner and evidence data which is objective biological information acquired from the patient. Here, a judgment sentence, which is a text sentence indicating judgment made in the medical-related work is used here as an example of information to be input by the medical practitioner. The evidence data indicates data such as laboratory data or image data acquired from a modality. Furthermore, workflow input means 107 configured to receive an input for selecting workflow step information from the medical information database 105, workflow step request input means 117 configured to receive an input of information on a request of the workflow step, input information accepting means 108 configured to accept an input of the judgment sentence, medical information output means 109 configured to extract and output the medical information from the medical information database 105 on the basis of the workflow step information, workflow output means 118 configured to acquire the workflow information from the medical information database 105 on the basis of a patient identifier for identifying the patient and output the same to the interface 111, and, workflow terminating means 119 configured to store workflow termination information in the medical information database 105 are provided. Functions of these means are stored in the memory 112 in FIG. 1A.

In this configuration, the medical information is displayed from the medical information database 105 on the basis of the workflow step information selected by the workflow input means 107, and the information input by the input information accepting means 108 and the displayed medical information are stored in the medical information database 105 in relation to the workflow step information being in execution. In the workflow step being in execution, by storing the referred medical information together with the input information, the information referred to and used as a foundation when executing the corresponding workflow step is accumulated, so that the processing of the medical-related work by the medical practitioner become clear.

The medical information database 105 accumulates and manages the medical information, which is information on the medical-related works in the respective workflow steps via the medical information storage means 106 and accumulates the judgment sentences input by the input information accepting means 108 in relation to the respective workflow steps, evidence data data-processed by evidence data processing means 115, the processing archival records thereof, and the medical information referred to in the respective workflow steps therein. As illustrated in FIG. 1A described above, the medical information database 105 may have link information to the medical information accumulated in the electronic health record system 102 and the PACS 103.

The medical information storage means 106 is means configured to store the workflow information, the workflow step information, and the medical information in the medical information database 105. For example, the medical information to be stored includes the evidence data data-processed by the evidence data processing means 115, the data processing archival record containing the data before processing thereof, information input by the medical practitioner by the input information accepting means 108.

The laboratory information database 116 accumulates and manages the information on the result of medical-related tests such as a blood screening or an imaging test in the respective workflow steps. The database may be realized by sharing database owned by a hospital information system such as the electronic health record system 102 and an ordering system or an image management system such as PACS 103 or the like, or data import means, not illustrated, may be provided to import data from these systems. It is also possible to provide data input means, not illustrated, and input the data directly by the doctor, the clinical nurse, or the engineers. The laboratory result of the patient based on a test method newly developed by a doctor for the purpose of study may be input from the data input means, not illustrated, and may be accumulated and managed together with general laboratory results. In addition, when the electronic health record system 102 or the PACS 103 is installed separately from the medical care support system 101, it is also possible to have only link information to laboratory information stored in the electronic health record system 102 or the PACS 103. When a data processing work station is installed, the archival record of the data processing executed in the data processing work station may be stored in the laboratory information database 116.

The evidence data processing means 115 is means configured to perform data processing by using the data accumulated in the laboratory information database 116. The evidence data processing means 115 includes a plurality of processing modules. The evidence data processing means 115 for doctors specialized in diagnostic imaging (hereinafter, referred to as a doctor interpreting radiogram) includes a basic module such as input-output processing of image data or various filtering processing and a functional module such as an area extractive processing and an image aligning processing having an advanced image processing algorithm. An operator combines the above-described processing modules freely depending on the object of the processing or characteristics of the data and performs a series of processing works required for the medical care on the data.

Here, the medical information database 105 and the laboratory information database 116 are logically discriminated. However, the physically identical database may be applied. For example, the medical information database 105 and the laboratory information database 116 may be combined to construct a single database. As illustrated in FIG. 1A, it is also possible to use the PACS 103 and the electronic health record system 102 separately and provide link information thereto.

In addition, the medical care support system of the present invention may have process analyzing means 160. The process analyzing means 160 is means for analyzing the process of the medical care on the basis of data of a plurality of patients accumulated in the medical information database 105 and extracting information required for improvement and optimization of the process for improving the quality and efficiency of the medical care.

<Description of Table Structure of Database>

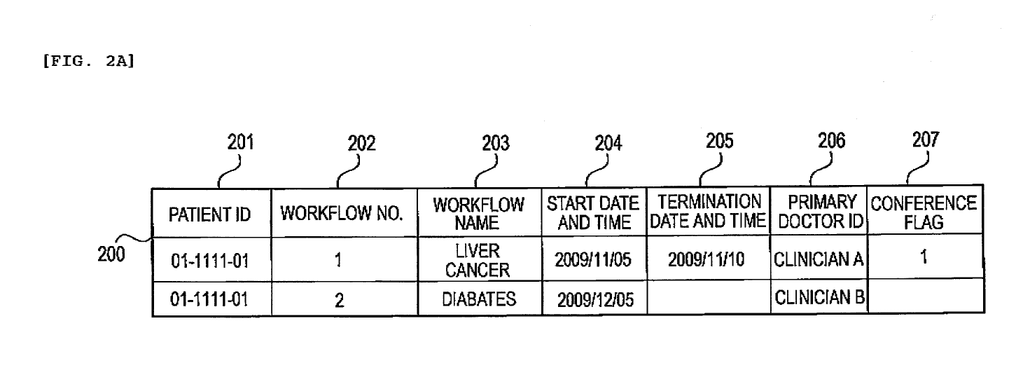

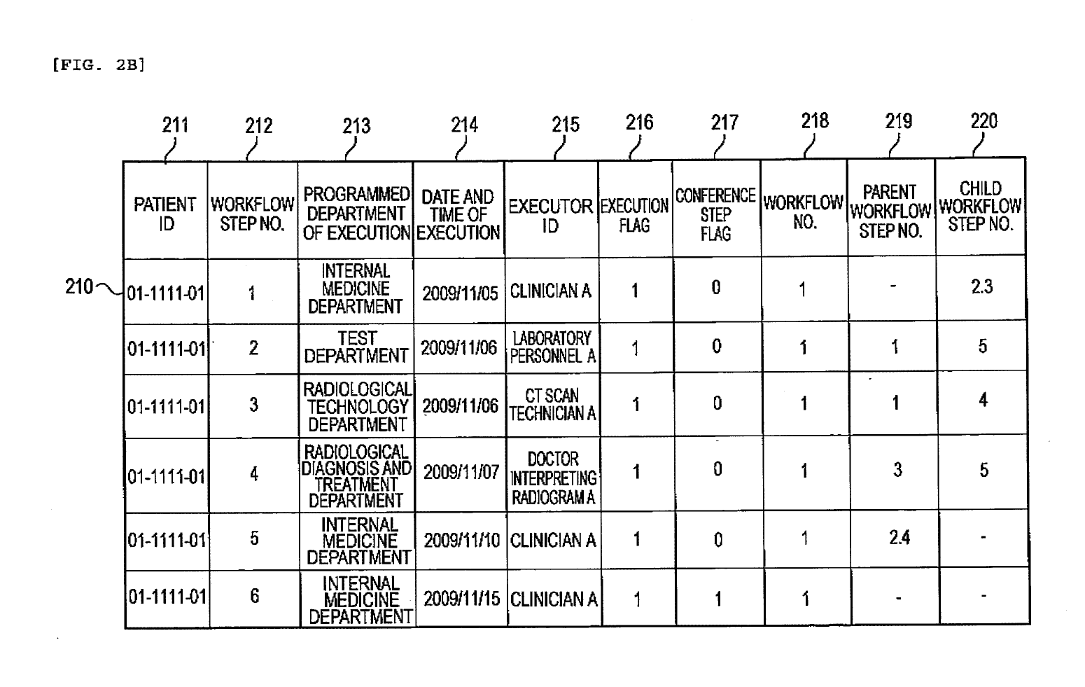

FIG. 2 illustrates a workflow information table 200 and a workflow step information table 210 included in the configuration of the medical information database 105.

The workflow information table 200 is a table for storing workflow information, which is information for identifying the workflow, and is basically registered by the primary doctor. A patient ID field 201, a workflow No. field 202, a workflow name field 203, a workflow start date and time field 204, a workflow termination date and time field 205, a primary doctor ID field 206, and a conference flag field 207 are included. The patient ID field 201 stores the patient identifiers, which are identifiers for identifying the patients. The workflow No. field 202 stores numbers which are key information for uniquely specifying the respective workflow information. The workflow name field 203 stores workflow names indicated by the names of diseases and the names of therapies or the like. The workflow start date and time field 204 stores the start days of the workflow. The workflow termination date and time field 205 stores the dates of termination of the workflows, which are registered at the time of terminating the workflows. The primary doctor ID field 206 stores identification information about the primary doctors, who have responsibility for the workflows. Here, although the workflow name field 203 stores the text sentences, it may store standard master IDs of the names of diseases or the names of therapies. The conference flag field 207 stores execution flags indicating that conferences have executed for the corresponding workflows.

The workflow step information table 210 is a table that stores workflow step information, which is information for identifying respective steps in the workflows, and one step corresponds to one record. The workflow step information table 210 includes a patient ID field 211, a workflow step No. field 212, a programmed department of execution of workflow step 1D field 213, a workflow step execution date and time field 214, a workflow step executioner ID field 215, a workflow step execution flag field 216, a conference step flag field 217, a workflow No. field 218, a parent workflow step No. field 219, and a child workflow step No. field 220. The workflow step No. field 212 stores numbers which are key information for uniquely specifying the respective workflow steps. The programmed department of execution of workflow step 1D field 213 stores identification information for uniquely identifying a programmed department, a diagnosis and treatment department, or a test department or the like in which the workflow steps are to be executed. The workflow step execution data and time field 214 stores the date and time when the workflow steps are executed. The workflow step executioner ID field 215 stores identification information for uniquely identifying medical practitioners who have actually executed the workflow steps. The workflow step execution flag field 216 stores flags indicating whether the workflow steps are executed or not. The conference step flag field 217 stores flags indicating that the workflow steps have taken to conferences. The workflow No. field 218 stores identification information for uniquely identifying the workflows which the workflow steps belongs to. Parent workflow step Nos. are identification information for identifying workflow steps located before the corresponding workflow steps and are coupled to the corresponding workflow steps. Child workflow step Nos. are identification information for identifying workflow steps located after the corresponding workflow steps and are coupled to the corresponding workflow steps. The parent workflow step Nos. and the child workflow step Nos. have a role to couple the client and the requested party of the order.

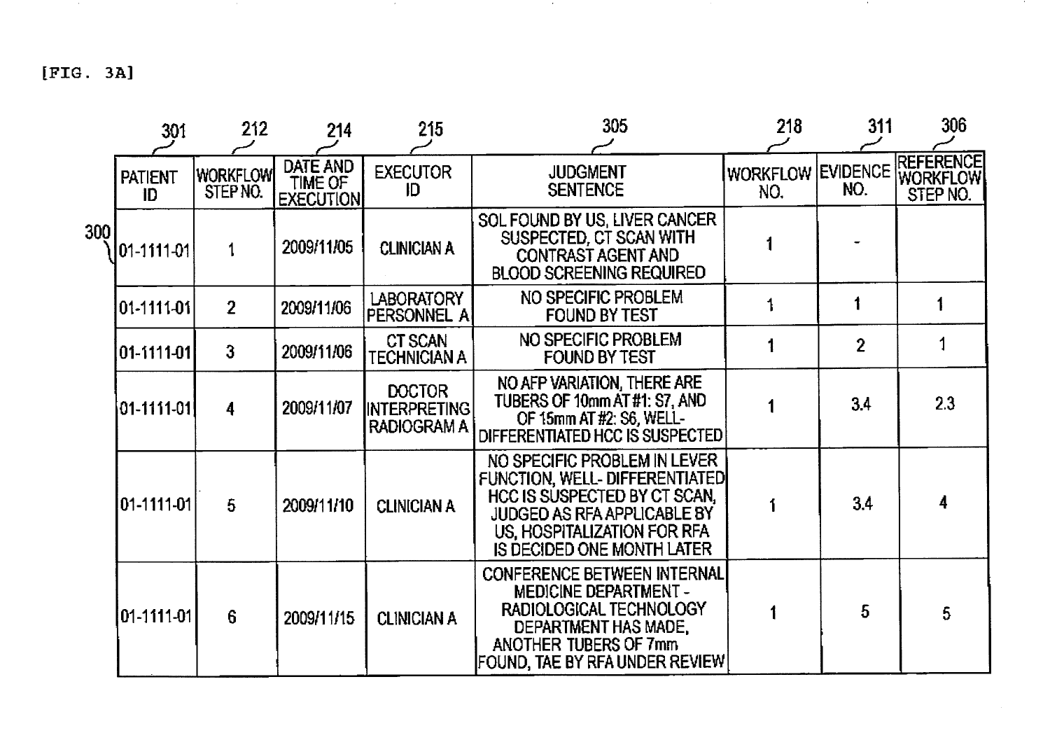

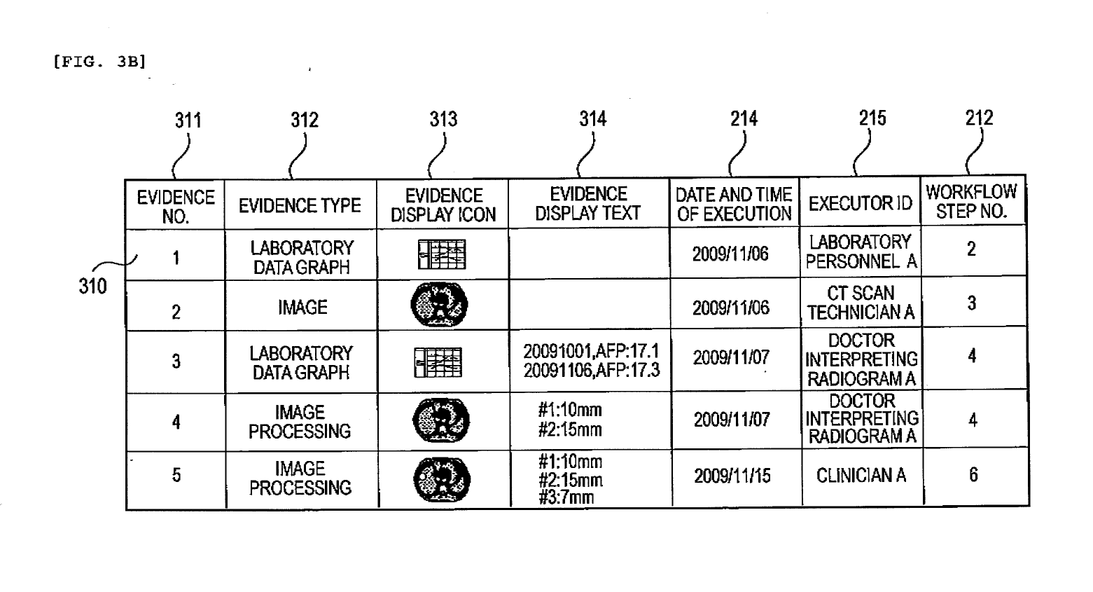

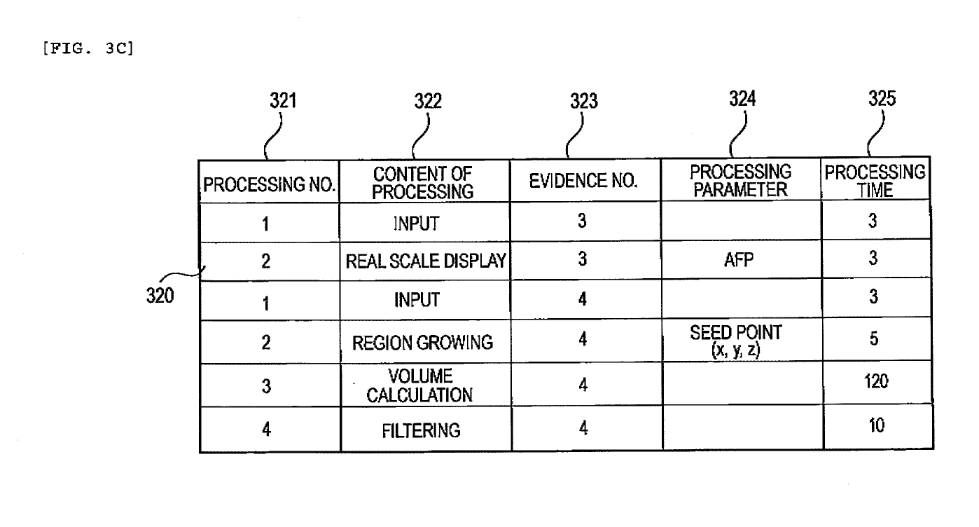

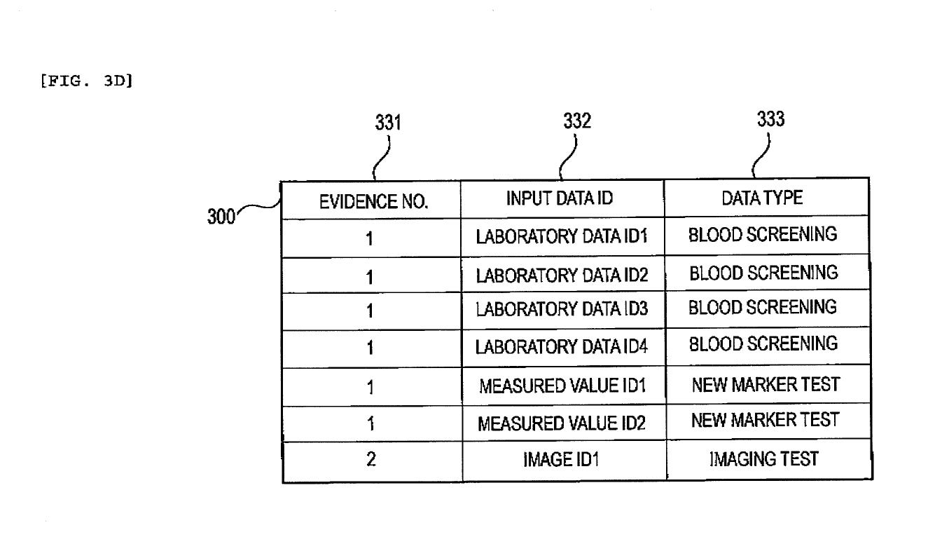

FIG. 3A illustrates a medical information table 300 contained in the configuration of the medical information database 105, and FIG. 3B illustrates an evidence data table 310. FIG. 3C illustrates a processing archival record table 320 also stored in the medical information database 105, and FIG. 3D illustrates an input data table 330.

The medical information table 300 is a table for storing medical information, which is information on medical-related works, on the basis of the workflow steps, and stores one item of medical information in one record. This table includes a patient ID field 301, the workflow step No. field 212, the workflow step execution date and time field 214, the workflow step executioner ID field 215, a judgment sentence field 305, the workflow No. field 218, an evidence No. field 311, and a reference workflow step No. field 306. The patient ID field 301 stores patient identifiers for identifying the patients. The judgment sentence field 305 stores judgment sentences input in the corresponding workflow step. When a judgment sentence is included in the medical information that is referred to when inputting the judgment sentence in the judgment sentence field 305, the judgment sentence referred to can be stored together. Identification between the input judgment sentence and the judgment sentence referred to is achieved on the basis of the reference workflow step No. The workflow step No. field 212 is stored when the medical information is registered, and stores link information to the workflow step No. field 212 in the workflow step information table 210. Accordingly, association between the workflow step information table 210 and the medical information table 300 is established. The evidence No. field 311 is an identifier for specifying evidence data which serves as grounds when making a judgment, and is associated with the evidence data table 310. In this manner, the judgment sentence and the evidence data which serves as the grounds of the judgment are stored in the medical information table 300 on the basis of the workflow step information. The reference workflow step No. field 306 stores workflow step Nos. referred to in the workflow step No. of the records.

The evidence data table 310 is a table for storing the evidence data, and stores one evidence data in one record. The evidence data table 310 includes the evidence No. field 311, an evidence type field 312, an evidence display icon field 313, an evidence display text field 314, the workflow step execution date and time field 214, the workflow step executioner ID field 215, and the workflow step No. field 212. The evidence No. field 311 stores numbers, which are key information for uniquely specifying the respective evidence data.

The evidence data processing means 115 performs different types of processing such as image processing for images, illustration in graphs or statistical processing for laboratory data depending on the type of the laboratory information to be input. Here, the evidence data for the different types of processing such as the image processing and illustration of the laboratory data in graphs are stored as different records, respectively. Then, the evidence type field 312 stores types of medical care support system from which the evidence data are extracted. The evidence display icon field 313 and the evidence display text field 314 store information which allows recognition of the contents of the evidence data. Icon images may be stored directly in the evidence display icon field 313 as illustrated in the drawing, and identification information of the icon images such as file names may be stored therein. The workflow step No. field 212 stores the link information to the workflow step No. field 212 in the medical information table 300. With this link, various data in the evidence data stored in the evidence data table 310 is in correspondence with the medical information table 300 via the link of the workflow step No.

As illustrated in FIG. 3C, the processing archival record table 320 is a table for storing the archival record of processing performed on the laboratory information, which is information before the processing of the evidence data, and one processing archival record is stored in one record. The processing archival record includes an archival record performed on the laboratory data and an archival record performed on the image data, and these processing archival records are included in the medical information. The processing archival record table 320 includes a processing number field 321, a processing content field 322, an evidence number field 323, and a processing parameter field 324. The processing No. field 321 stores numbers, which are key information for uniquely identifying respective processing items which constitute the processing archival record. The processing content field 322 stores information minimum required for a calculator to reproduce, analyze, and reuse the processing later, that is, information which allows identification of the processing contents. In FIG. 3C, information on the processing archival record of the image processing and the laboratory data processing is illustrated. In laboratory data processing (evidence No.=3), a case where the laboratory results are read to illustrate a plurality of test items in graphs in a real scale is shown, and “input” and “real scale display” are registered. In the image processing (evidence No.=4), a case where an image to be tested is read to perform area extracting processing on a tumor-included area (here, for example, a region glowing method) and calculate the volume of the tumor-included area is shown, and are registered “input”, “region glowing”, and “volume calculation” in the order of processing items. The evidence No. field 323 stores values of the evidence No. field 311 in the evidence data table 310. It indicates that the processing is executed on the evidence data identified by the same evidence No. in the order of the number of the processing No. field 321 or the like. The processing parameter field 324 shows parameters set when executing the respective processing items. Although it is put in one field in this example for the sake of convenience of explanation, it depends on the processing items. Therefore, different processing parameter tables may be prepared for the respective types of the processing. In the configuration of this example, the individual processing items which constitute the evidence data is saved in the table of the database individually. However, in the evidence data table 310, it is also possible to provide a processing archival record binary field and store the same including the processing archival record and the input-output data of the respective processing items in its own binary form. Accordingly, when reproducing the processing, speed-up of the reproduction of the processing and easiness of mounting are achieved by passing the binary data directly to the evidence data processing means 115 without referring to the processing archival record table 320 and the input data table 330. In this manner, in the processing archival record table 320, the contents of the processing or the like are associated with the records stored in the evidence data table 310 and the medical information table 300 via the evidence Nos.

The input data table 330 is linked with the laboratory information database 116, stores input data to be used in the first processing in the processing archival record in the evidence data, and stores one input data in one record. The input data table 330 includes an evidence No. field 331, an input data ID field 332, and an input data type field 333. The input data ID field 332 stores numbers or the like which are key information for uniquely specifying the laboratory information in the laboratory information database 116 as input data for the respective evidence data. The input data type field 333 stores data types for setting down linked destinations of IDs stored in the input data ID field 332. For example, when it is a “blood screening”, the input ID is a laboratory data ID for uniquely identifying the laboratory data of the patient, when it is an “imaging test”, it is an image ID for uniquely identifying the image of the patient, and when it is a “new marker test”, it is an measured value ID for uniquely identifying the measured value of the patient to be subjected to the new marker test. In this manner, the input data table 330 is associated with the records stored in the evidence data table 310 and the medical information table 300 via the evidence No. field 331.

<Laboratory Information Database>

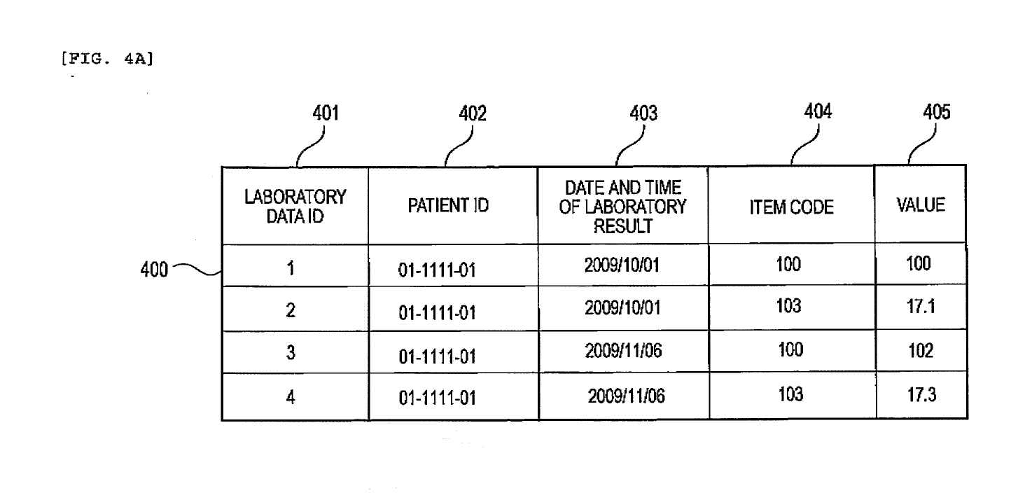

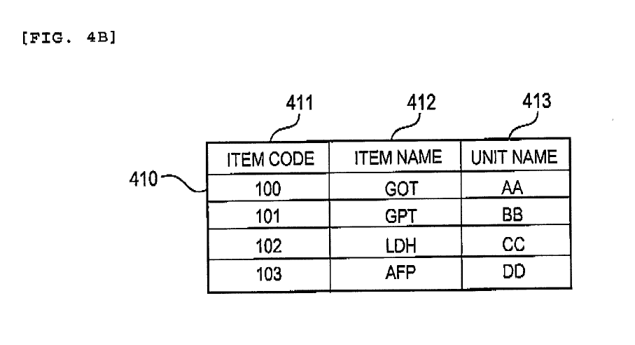

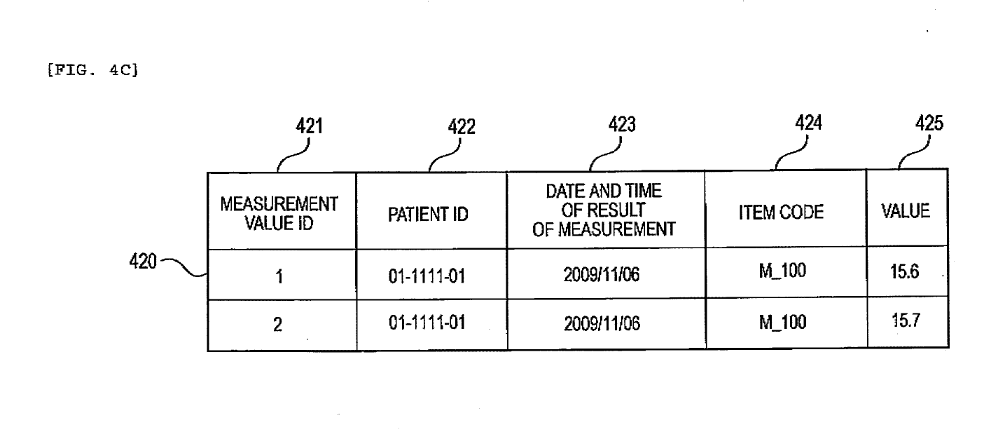

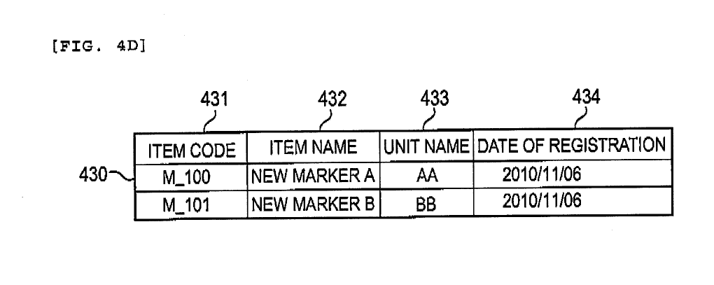

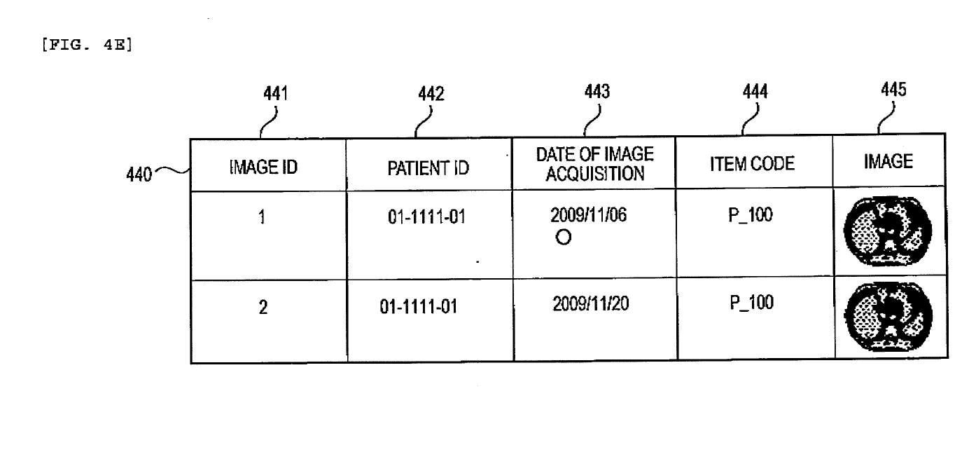

FIG. 4A to FIG. 4E illustrate an example of a table group which constitutes the laboratory information database 116. The laboratory information database 116 stores detailed information of the evidence data relating to the medical cares, and includes a laboratory data table 400 (FIG. 4A), a test item master table 410 (FIG. 4B), a measured value table 420 (FIG. 4C), a measurement item master table 430 (FIG. 4D), and an image table 440 (FIG. 4E). Here, detailed description about the respective tables will be given. The laboratory data table 400 in FIG. 4A is a table for storing the content of the “blood screening” data, and stores one laboratory data in one record. The laboratory data table 400 includes a laboratory data ID field 401, a patient ID field 402, a laboratory result date and time field 403, an item code field 404, and a value field 405, and is associated with the test item master table 410 with an item code as a key. The test item master table 410 in FIG. 4B is an item master table relating to the “blood screening”, and includes an item code field 411, an item name field 412, and a unit name field 413. The measured value table 420 is a table for storing the content of “new marker test” data, and stores one measured value in one record. The measured value table 420 in FIG. 4C and the measurement item master table 430 in FIG. 4D are configured of the same structure as the above-described laboratory data table 400 and the above described test item master table 410. A measured value ID field 421, a patient ID field 422, a measured result date and time field 423, an item code field 424, and a value field 425 are included in the measured value table. The measurement item master table 430 includes an item code field 431, an item name field 432, and a unit name field 433. The measurement item master table 430 may be provided with a field for storing information on registration of the measurement item (for example, date of registration field 434 and the like) in addition to the fields which constitute the test item master table 410 to allow a version management of the item.

Here, as described thus far, the content of the input data in the laboratory information database 116 manages the “blood screening” and the “new marker screening” separately, and in the input data table 330 is configured to manage only link information in an integrated fashion. Accordingly, since the laboratory data measured in the normal work and the laboratory data obtained by a newly developed laboratory method for the purpose of research may be accumulated as the evidence data in the same manner, analysis of the effectiveness of the newly developed laboratory method is enabled.

The image table 440 in FIG. 4E includes an image ID field 441 configured to store an identifier for identifying an image, a patient ID field 442 configured to store an identifier of a patient, an image acquisition date field 443 configured to store date and time when the image is acquired, an item code field 444 configured to store the item code, and an image field 445 configured to store the image.

<Example of Doctors Interpreting Radiogram>

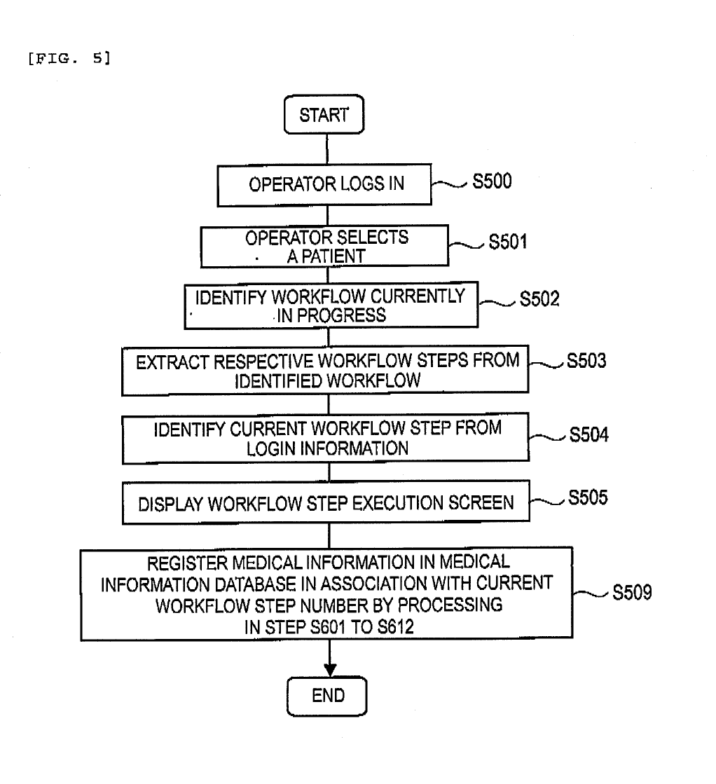

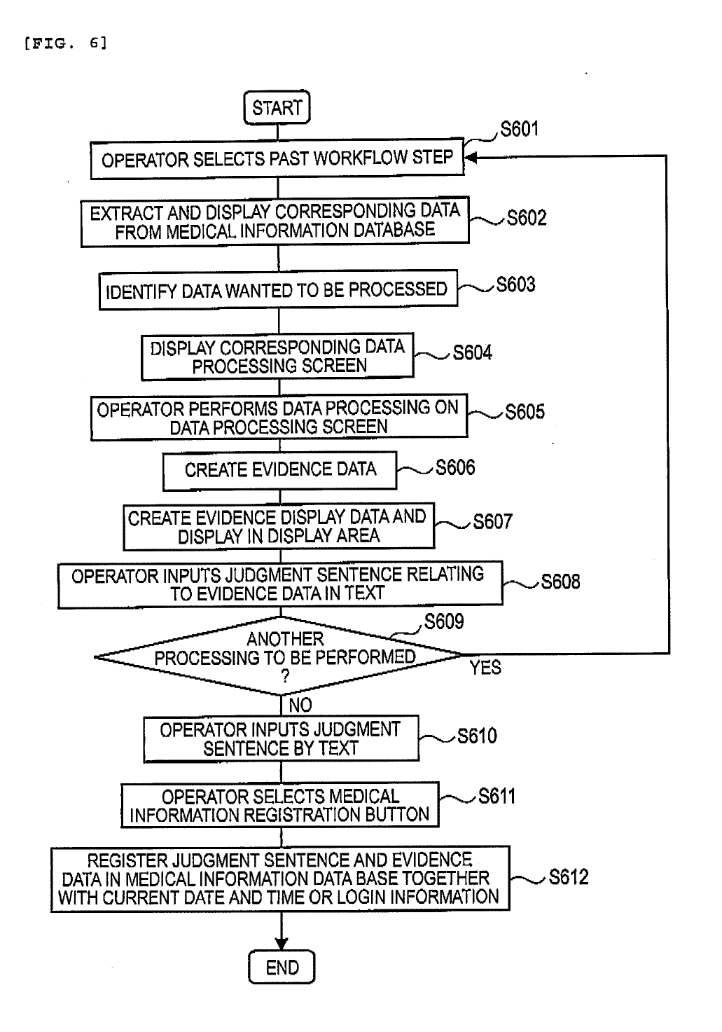

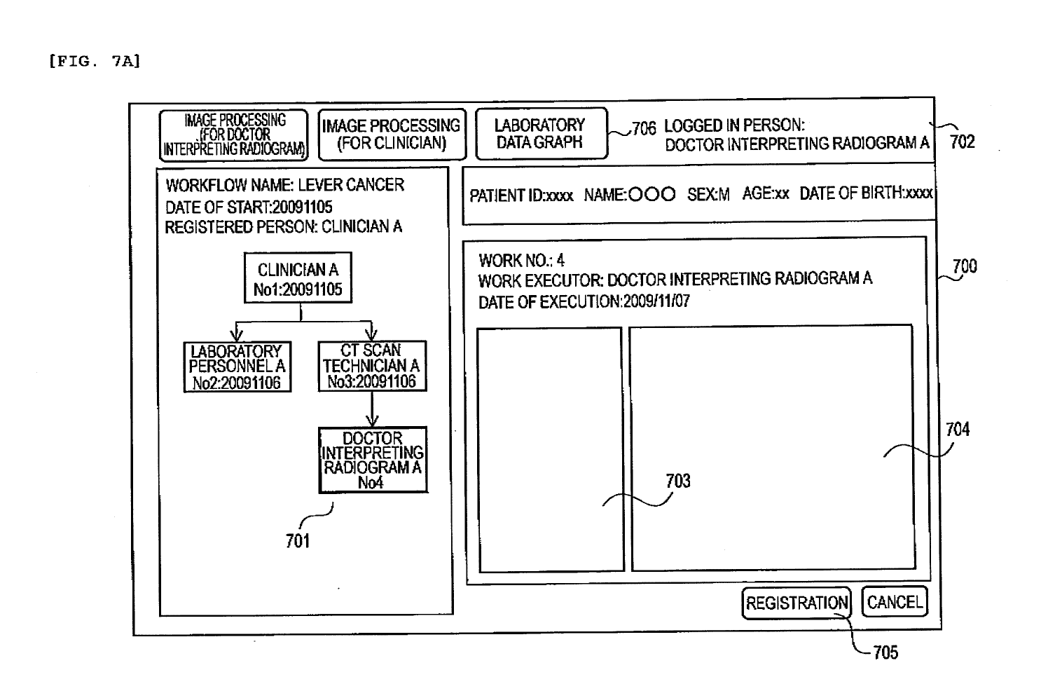

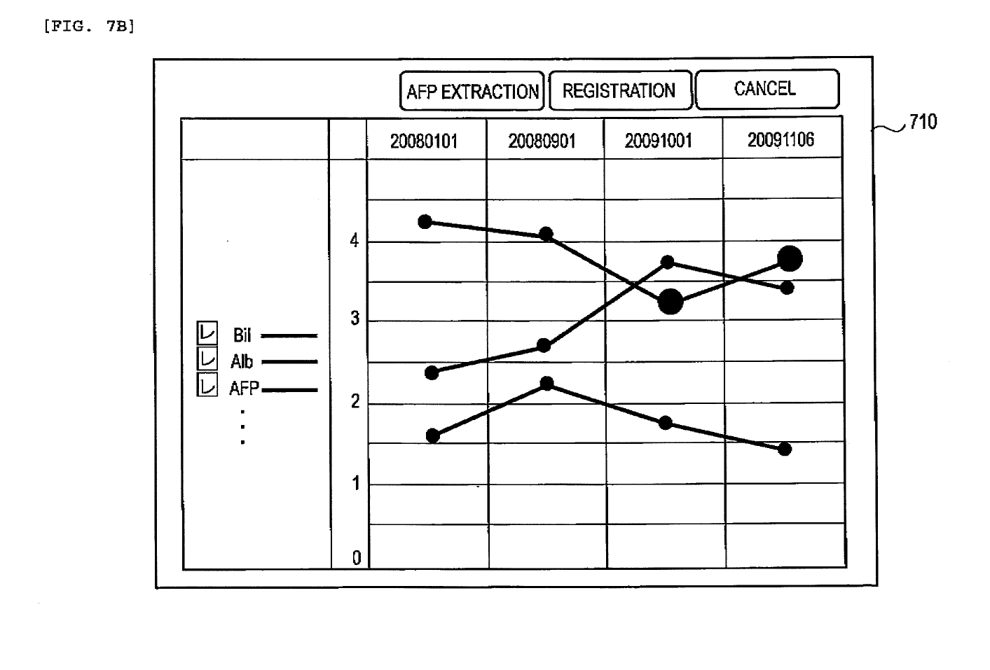

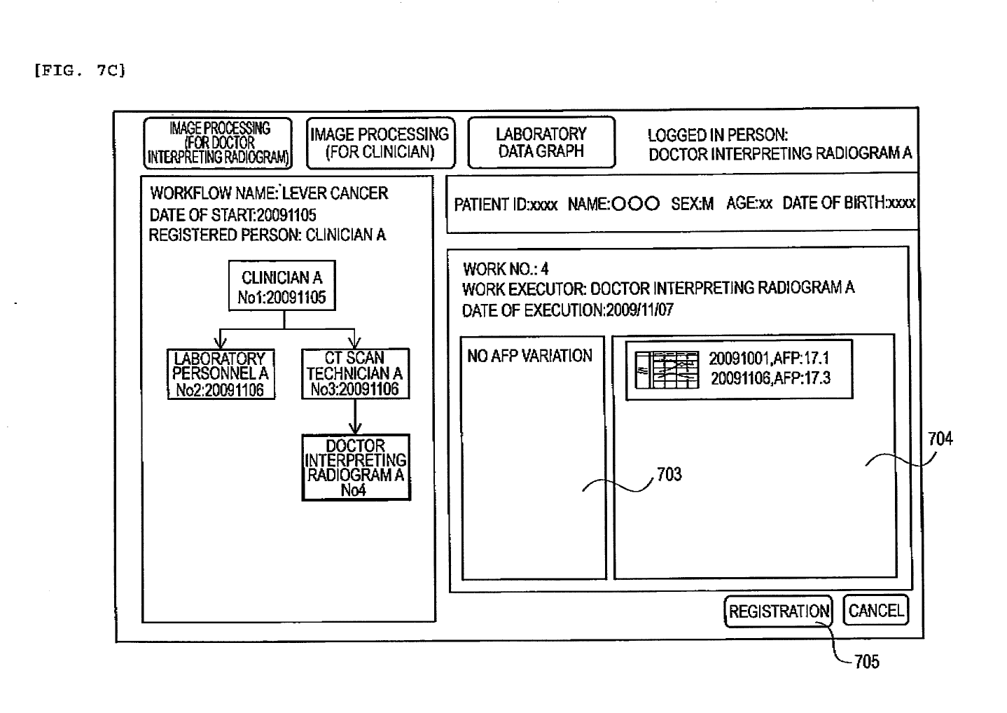

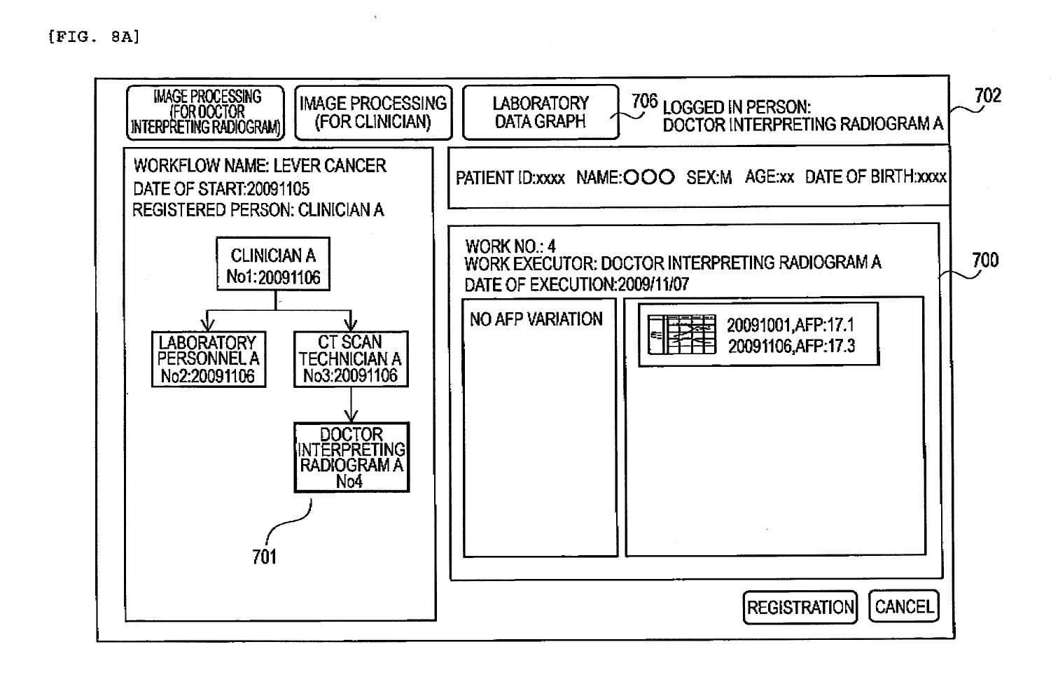

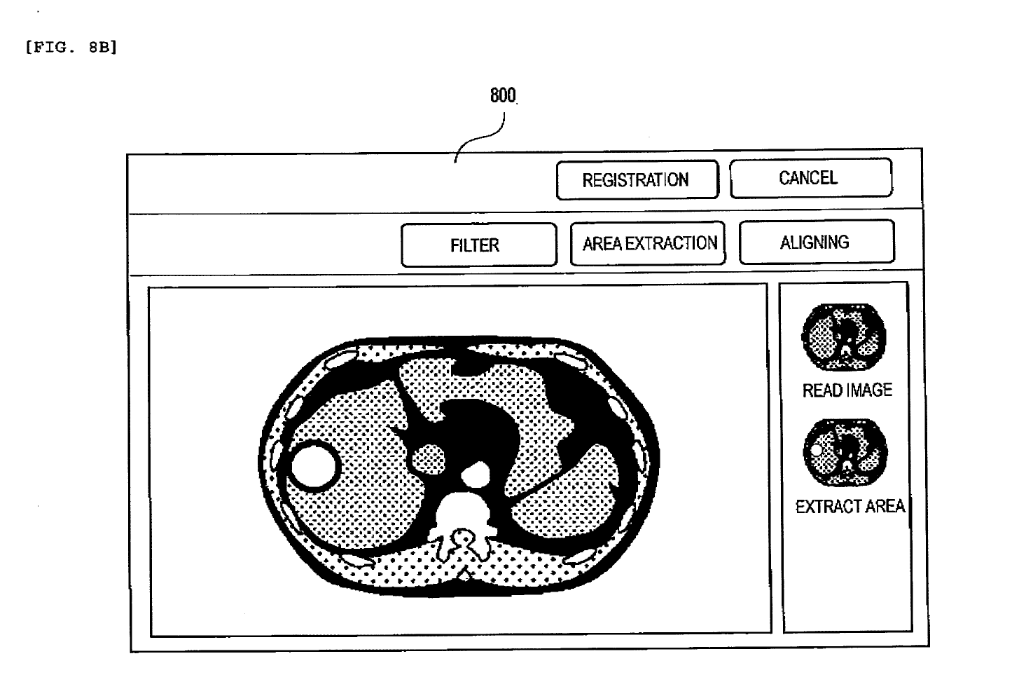

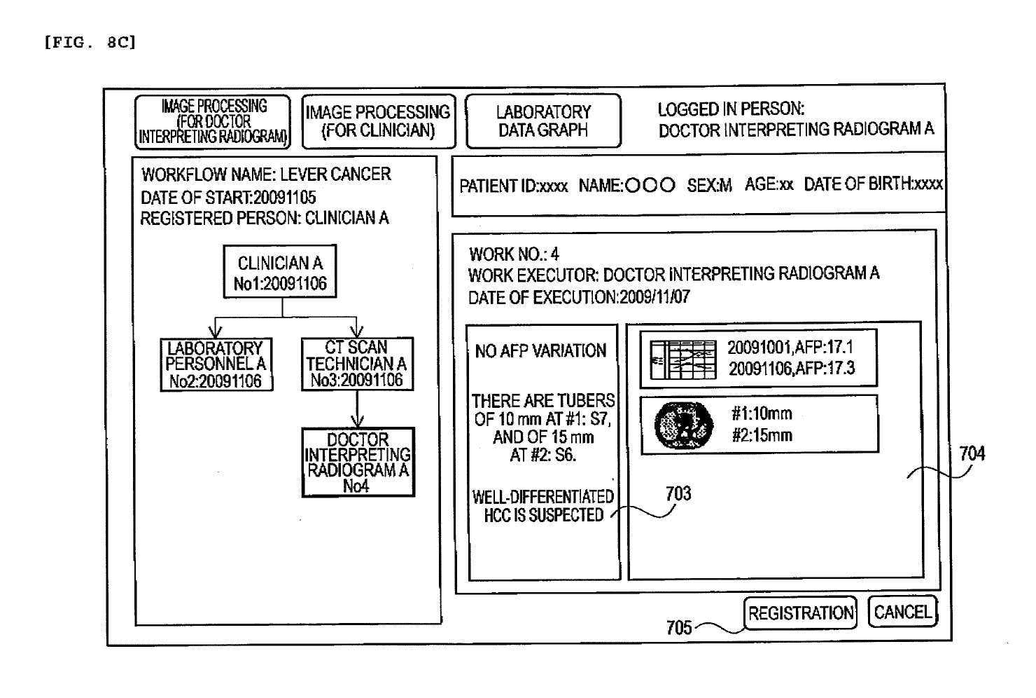

Subsequently, a data input operation of this system will be described in detail by using processing flows illustrated in FIG. 5 and FIG. 6 and display examples illustrated in FIG. 7A to FIG. 7C and FIG. 8A to FIG. 8C.

In this system, the terminal 104 accepts a login input by the operator via a login screen (Step S500). Subsequently, when the terminal 104 accepts an input of selecting a patient identifier of one patient by the operator via a patient selecting screen (Step S501), the medical information storage means 106 identifies a workflow No. being currently in progress by using a record corresponding to the patient acquired in Step S501 from the workflow information table 200 in FIG. 2. Here, a method of identifying the workflow No. being currently in progress may be, for example, a method of identifying a record in which a value of the workflow termination date and time field 205 is not registered yet, or a method of extracting all the records corresponding to the patient acquired in Step S501 from the workflow information table 200, displaying a workflow selecting screen, and specifying one workflow by the operator.

The medical information storage means 106 acquires corresponding workflow step information from the workflow step information table 210 in FIG. 2 from the workflow No. identified in Step S502 (Step S503). A current workflow step No. is identified from department information in login information acquired in Step S500 (Step S504). A current workflow step indicates a workflow step being currently in execution in the workflow. The identification of the current workflow step is achieved, for example, by the medical information storage means 106 referring to the programmed department of execution of workflow step 1D field 213 and the workflow step execution flag field 216 in the record acquired in Step S502 and extracting a department of the logged-in person and a workflow step No. which is not executed in a step of the corresponding department as a current workflow step No. When everything has already executed, the last step No. is assigned as the current workflow step No. Subsequently, the workflow output means 118 sets and displays respective workflow step information acquired in Step S503 on a workflow step execution screen 700 illustrated in FIG. 7A (Step S505). The workflow step execution screen 700 is illustrated in the interface 111 illustrated in FIG. 1A.

The workflow step execution screen 700 in FIG. 7A includes, for example, a login information display area 702, a workflow step selection area 701, a judgment sentence I/O area 703, an evidence data display area 704, a medical information registration button 705, and a medical care support button group 706 as illustrated in the drawing. The workflow step selection area 701 displays workflow steps from a start step to a current step of the workflow on the basis of the information of the parent workflow step No. field 219 of the workflow step information table 210 in a flow form. For example, as illustrated in the workflow step information field 210 in FIG. 2B, workflow steps whose workflow step Nos. are 2 and 3, whose parent workflow step No. being 1, are displayed in connection with the workflow step whose workflow step No. is 1, and a workflow step whose workflow step No. is 4, whose parent workflow step being 3, is connected to the workflow step whose workflow step No. is 3. The same connection may be made also with the child workflow step No. In this manner, the workflow information and the workflow step information are displayed on the workflow step selection area 701 in FIG. 7A so as to connect the client and the requested party.

The login information display area 702 is an area for displaying information on the operator logging in the current system. The judgment sentence I/O area 703 is an area for displaying input and output of the content judged by the medical practitioner in a text form. The evidence data display area 704 displays evidence data. The medical information registration button 705 is a button for registering a combination of the evidence data and the text sentence in the medical information database 105 by a clicking action of the operator. The medical care support button group 706 is a button to be used by the medical practitioner for calling up a function of the evidence data processing means 115 configured to perform the data processing such as the image processing or the laboratory data processing for a medical care of the patient. The medical care support button group 706 may be configured to be capable of setting “selectable” or “non-selectable” from information on a job type in the login information acquired in Step S500.

Here, in the workflow step selection area 701, for example, the current workflow step identified in Step S504 is highlighted. Also, information which indicates a progress situation of the workflow is displayed in the respective workflow steps. In FIG. 7A, a display form which indicates whether the steps are completed or not, such as changing a display color in accordance with the content of the workflow step execution flag field 216, for example, is taken. Also, as regards the completed workflow step, contents of the workflow step execution date and time field 214 and the workflow step executioner ID field 215 are displayed.

Here, it is assumed that a doctor interpreting radiogram A proceeds to a radiogram interpreting work, and registers medical information. The doctor interpreting radiogram A performs the radiogram interpreting work by processing in Steps S601 to S612 in FIG. 6 (described later). When the radiogram interpreting work is terminated, the medical information storage means 106 registers medical information displayed in correspondence with the current workflow step No. identified in Step S504 to the medical information database 105 (Step S509).

FIG. 6 illustrates detailed actions of the system when registering the medical information. Here, an example in which the radiogram interpreting is performed while referring to medical information in the workflow step of laboratory personnel A or a CT scan technician A in the workflow step of the radiogram interpreting work, and the medical information referred to and the result of radiogram interpreting are saved in association with the workflow step being in execution with this system will be shown.

First of all, the operator inputs a selection of workflow step information of the laboratory personnel A (Step S601). The workflow input means 107 accepts the input of the selection of the workflow step information from the medical information database 105. The medical information output means 109 searches medical information associated with the selected workflow step (workflow step No. 2) of the laboratory personnel A from the medical information database 105, and displays the same on the workflow step execution screen 700 (Step S602). It is assumed here that a laboratory data graph of the evidence No. 1 acquired by the laboratory personnel A is registered in the medical information. The doctor interpreting radiogram A specifies the displayed laboratory data graph as data on which execution of data processing is wanted (Step S603). A laboratory data graph display screen 710 as illustrated in FIG. 7B is displayed (Step S604). In the laboratory data graph display screen 710, selection of the test items to be displayed, setting of a graph form, and input of data processing such as extraction of point of fixation or data variations are accepted (Step S605). It is assumed here that data processing such as selecting and extracting a value of AFP called “AFP extraction” is performed. The evidence data processing means 115 extracts the value of the AFP selected by the operator from the AFP data in the laboratory data graph by the “AFP extraction”. In FIG. 7B, selected AFP data is indicated by enlarging dots indicating selected two points. The AFP value and date extracted in Step S605 are acquired as the evidence data together with the displayed laboratory data graph (Step S606). The medical information output means 109 creates evidence data for display “graph icon file 1”, “20091001, AFP: 17.1, 20091106, AFP: 17.3” from the acquired data, and displays the same in a first row of the evidence data display area 704 as illustrated in FIG. 7C (Step S607). Subsequently, the operator inputs a judgment sentence “AFP No Change” relating to the data processing of the laboratory data graph in the judgment sentence I/O area 703 (Step S608). The input information accepting means 108 accepts the input.

When the operator determines whether or not another data processing is to be performed (Step S609) and, when performing another data processing, the procedure goes back to Step S601, whereby the processing is repeated. In the case of this example, it is assumed that the operator performs the image processing on a CT image taken in the workflow step of a CT scan technician A in the screen illustrated in FIG. 8A (=FIG. 7C). The workflow input means 107 accepts an input for selecting a workflow step of the CT scan technician A (workflow step No. 3). In Step S5602, the image data (evidence No. 2) is acquired and displayed. Displayed image data is specified as data wanted to be subjected to the processing (Step S603). In Step S604, an image processing screen 800 as illustrated in FIG. 8B is displayed. In Step S605, the doctor interpreting radiogram A performs region extraction of a tumor with respect to the CT image. Detailed processing in the image processing screen 800 will be described later. In Step S606, an image processing archival record from input of an image until the calculation of the volume of the tumor region via the region extraction and an input data “image ID1” are acquired from the image processing screen 800. In Step S607, evidence data for display “image icon file 1” “#1: 10 mm #2: 15 mm” is created by using the information of the image processing archival record and is displayed on the evidence data display area 704 as illustrated in FIG. 8C. In Step S608, the operator inputs a judgment sentence “there are tubers of 10 mm at #1:S7, and of 15 mm at #2:S6” relating to the image processing in the judgment sentence I/O area 703. The input information accepting means 108 accepts this input.

Subsequently, when the operator judges not to perform additional processing in Step S609, a judgment sentence not for each of the evidence data, a judgment sentence, for example, “well-differentiated HCC is suspected” is additionally input in the judgment sentence I/O area 703 as needed (Step S610). The input information accepting means 108 accepts this input. Finally, the operator selects the medical information registration button 705 (Step S611). When the medical information registration button 705 is selected, the medical information storage means 106 acquires current date and time, for example, from hardware on which the medical care support system is mounted, and the medical practitioner information “doctor interpreting radiogram A” of a logged-in user is acquired, for example, from the medical information database 105. The medical information storage means 106 registers a text sentence of the judgment sentence I/O area 703 and an archival record and input data of the processing extracted by the evidence data processing means 115 in respective tables in the above-described medical information database 105 together with the patient identifier, the date and time, and the medical practitioner information selected in Step S501, a workflow step execution flag “REAL”, and the workflow step No. (Step S612). In this example, the evidence No. 3 is registered in response to the data-processed laboratory data graph and the evidence No. 4 is registered in response to the image-processed data, respectively.

In the judgment sentence I/O area 703 in this example, the text sentences input and output in Step S608 and Step S610 are treated as one data. However, it is also possible to configure the judgment sentence I/O area 703 to be separately include I/O areas (a judgment sentence area of the laboratory data graph, a judgment sentence area of the image processing), and an I/O area of the judgment sentence relating to all the evidence data, and register the respective judgment sentences in distinction from each other by adding tag information for distinguishing these areas and the like in Step S612. The judgment sentences for each of the respective evidence data may be provided with link information with respect to the evidence data.

In this manner, the processing of execution of the medical-related work by the medical practitioner indicating data referred to and a way how the judgment is made in the respective workflow steps of the corresponding medical practitioner may be accumulated in correspondence with the workflow step by displaying the evidence data corresponding to the medical-related work in the workflow step executed before the current workflow step, performing the data processing, accepting an input of a judgment sentence on the basis of the data-processed evidence data in the current workflow step, and registering the judgment sentence and the evidence data displayed at the time of input of the judgment sentence in correspondence with the current workflow step. Accordingly, medical care may be supported while utilizing an implicit knowledge of the respective medical practitioners along the workflows. In addition, by saving the processing archival record of the evidence data on the basis of the workflow step, the correspondence between the judgment of the medical practitioners and the data processing archival records in the respective workflow steps may be accumulated.

<Analysis Example 1 of Medical Care Process>

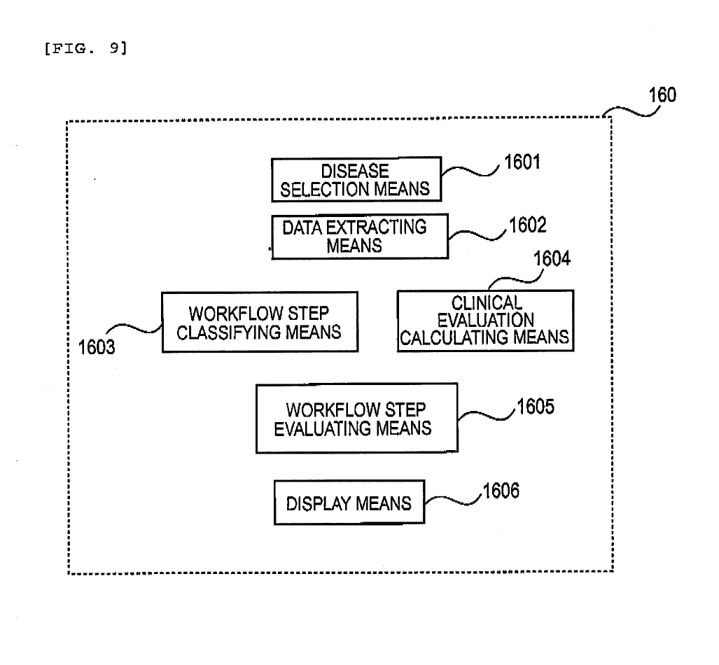

Subsequently, the process analyzing means 160 will be described in detail by using example of screens from FIG. 9 to FIG. 17. This system is characterized in that a clinical indicator (also referred to as Quality Indicator) which is an indicator evaluating the quality of medical treatment quantitatively for supporting the evaluation of the individual medical-related work called a workflow step. In other words, a workflow step performed when the evaluation value of the clinical indicator is high is executed to be highly acclaimed.

FIG. 9 illustrates an example of first configuration means of the process analyzing means 160. The process analyzing means 160 illustrated in FIG. 9 includes disease selection means 1601, data extracting means 1602, workflow step classifying means 1603, clinical evaluation calculating means 1604, workflow step evaluating means 1605, and display means 1606. Here, correspondence with the hardware configuration illustrated in FIG. 1A will be described. The process analyzing means 160 realizes various processing by deployment and activation of predetermined programs in the CPU 114, the memory 112, the memory device 113, or the like. Also, the disease selection means 1601 is operated on the basis of a signal input via the interface 111. An image configuration created in the display means 1606 is displayed on the terminal 104.

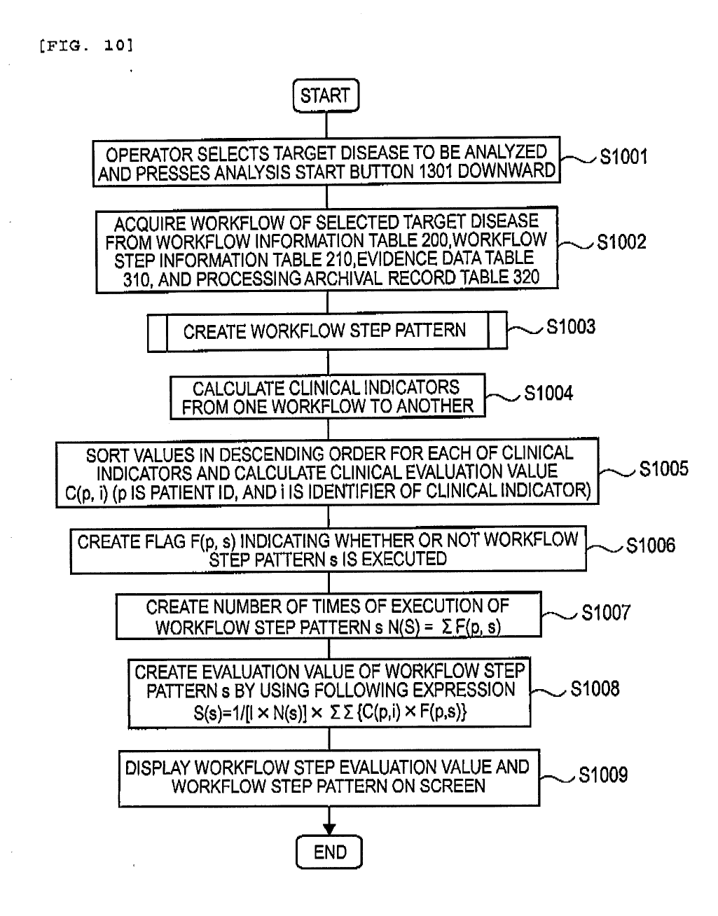

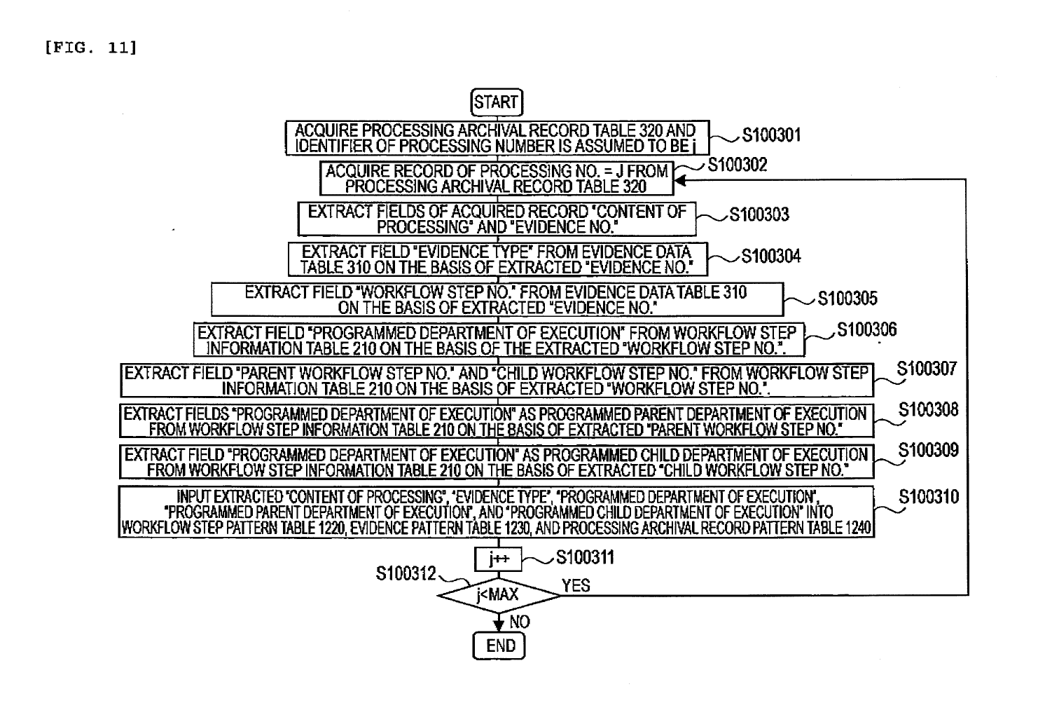

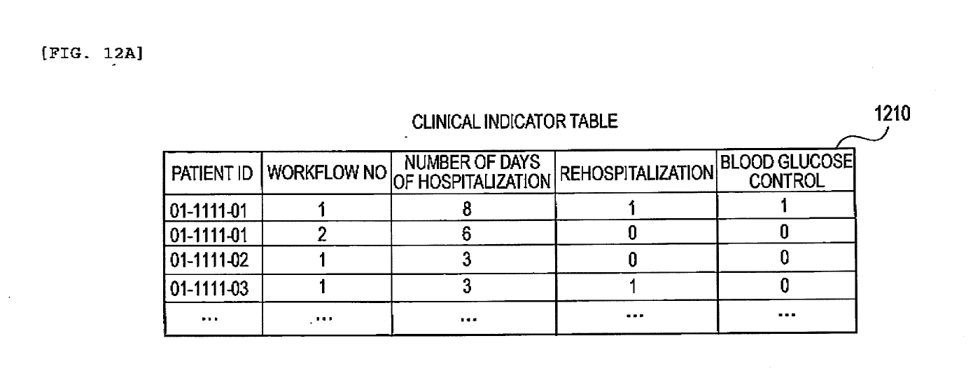

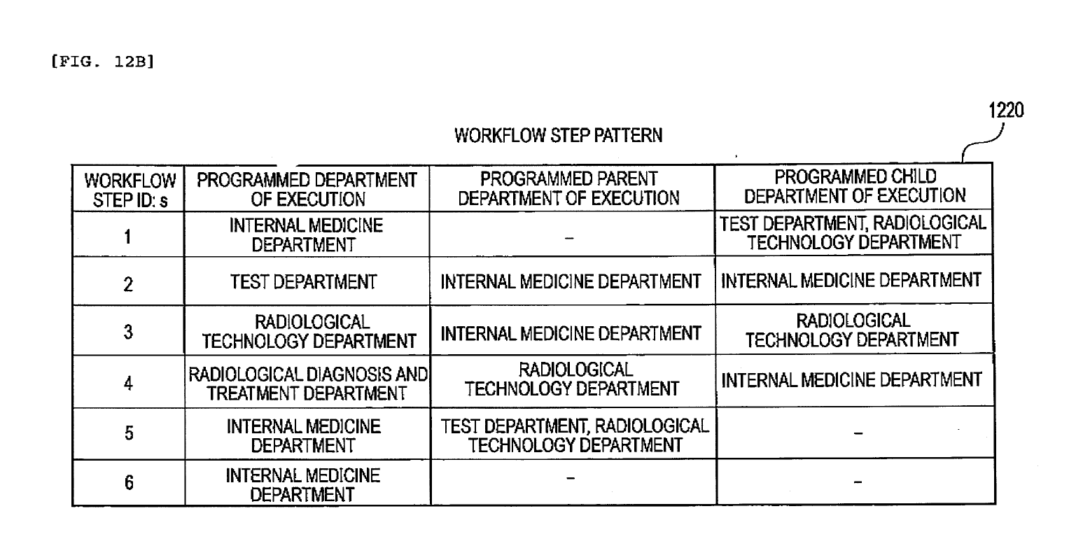

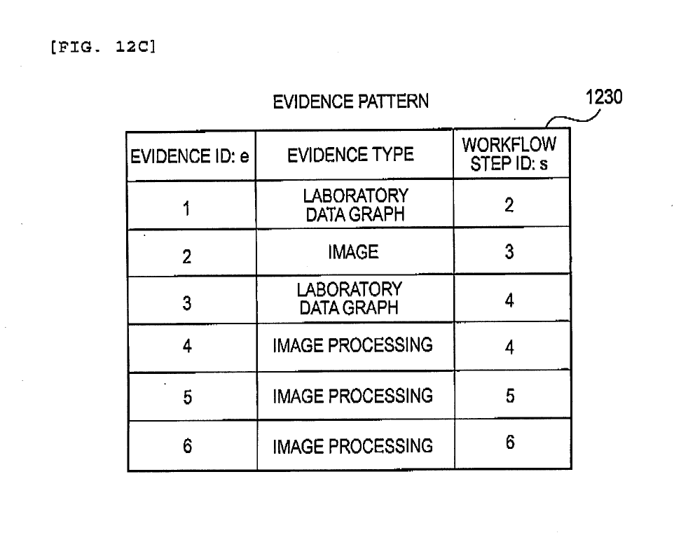

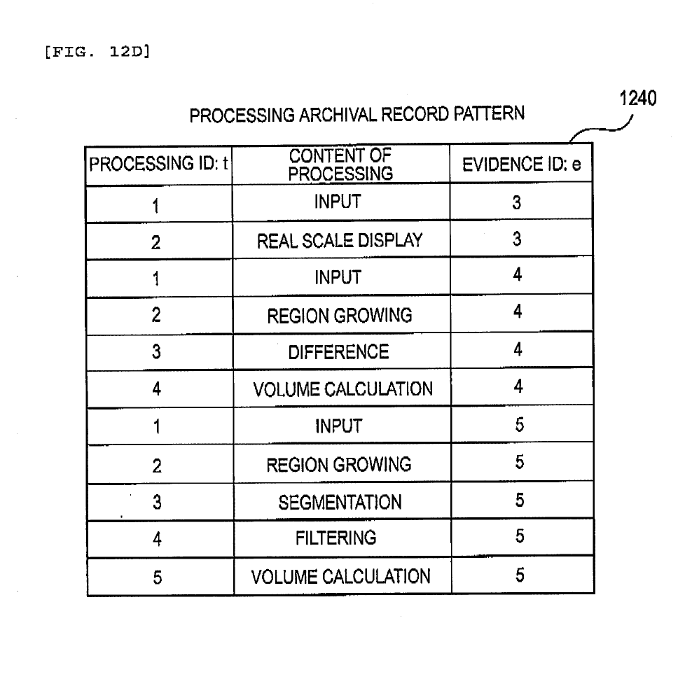

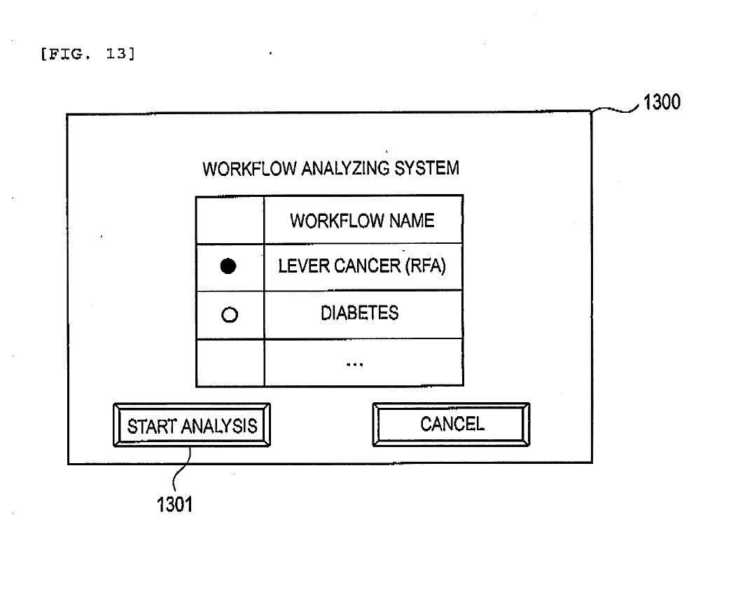

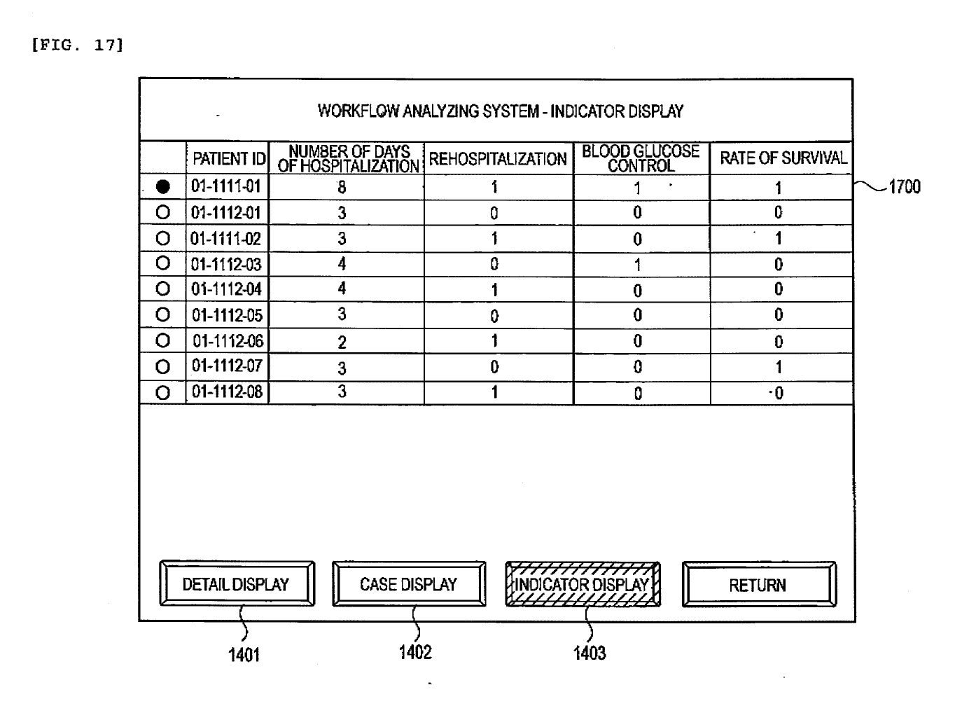

FIG. 10 illustrates an example of a first flowchart showing an outline of an operation of the process analyzing means 160. First of all, when the operator selects a disease to be analyzed via the interface 111, the disease selection means 1601 accepts an input for selecting the disease to be analyzed, and memorizes the selected disease to be analyzed (S1001). Subsequently, in the data extracting means 1602, the workflows having the name of the memorized disease to be analyzed and various items of information associated with the workflows are acquired from the workflow information table 200, the workflow step information table 210, the medical information table 300, the evidence data table 310, and the processing archival record table 320 stored in the medical information database 105 (S1002). Subsequently, in the workflow step classifying means 1603, the workflow steps as the records in the workflow step information table 210 from the acquired table are classified into partial sets having common medical-related work, and creates a workflow step items to be evaluated, that is, a workflow step pattern (S1003). In the following description, the workflow step item is referred to as the workflow step pattern. The detailed description of the classification will be given with reference to FIGS. 11 and 12. Subsequently, the clinical indicators as indicators obtained by measuring the qualities of the medical treatment from one patient to another are calculated, and the clinical evaluation values of the workflows are calculated are calculated on the basis of the clinical indictors by the clinical evaluation calculating means 1604 (S1004, S1005). Subsequently, the evaluation values of the respective workflow step patterns are calculated by the workflow step evaluating means 1605 on the basis of the values obtained by the workflow step classifying means 1603 and the clinical evaluation calculating means 1604 (S1006 to S1008). Finally, the workflow step pattern and a screen presenting the evaluation values thereof are configured in the display means 1606 (FIG. 13 o FIG. 17), and are displayed on the terminal 104.

Detailed descriptions about the workflow step classifying means 1603, the clinical evaluation calculating means 1604, and the workflow step evaluating means 1605 will be given below.

First of all, the object of the workflow step classifying means 1603 in the analysis example 1 will be described. In this system, in order to evaluate the workflow step executed when the evaluation value of the clinical indicators is higher than that of others to be highly acclaimed, the common medical-related work executed among different patients is extracted and how the clinical evaluation value varies is evaluated depending on whether or not the focused medical-related work is performed in the workflow step evaluating means 1605. Then, the common medical-related work is extracted. In general, there is a clinical path describing a standard medical act when performing a medical care on a certain disease, and extraction of the common medical-related work on the basis of the clinical path is conceivable. On the basis of this point of view, if the standard workflow is described in advance and the common medical-related work is extracted on the basis of the standard workflow, correspondence between the respective records of the workflow step information table 210 and the standard workflow may be extracted. However, an enormous labor and a sufficient experience are required for describing the standard workflow, and the progress of the medical knowledge is remarkable. Therefore, the maintenance work in the standard workflow also requires an enormous labor and lays a significant burden on many medical institutions, and hence it is not realistic. Accordingly, the workflow step classifying means 1603 extracts the common medical-related work from the accumulated data and creates a pattern of the workflow step.

FIG. 11 is an example of the first flowchart illustrating an operation of workflow step classifying means 1603 and is a detailed flowchart of S1003. In this flowchart, among a plurality of the workflow steps, the workflow step pattern are created for respective workflow steps in which the record of the evidence data table 310 and the record of the processing archival record table 320 are common and the parent workflow step No. field 219 and the child workflow step No. field 220 of the workflow step information table 210 are common. The reason why the parent workflow step No. field 219 and the child workflow step No. field 220 are added is that a series of flows of the medical-related work which is a characteristic of the workflow is to be considered.

The flowchart in FIG. 11 will be described in detail. First of all, the workflow step classifying means 1603 provides all the records of the processing archival record table 320 with identifiers j of the processing Nos. (S100301), and the following processing is performed with respect to the respective records (S100302 to S100311). First of all, the fields such as “content of processing” and “evidence No.” are extracted (S100303), and are output to a processing archival record pattern table 1240 stored in the medical information database as illustrated in FIG. 12D (S100310). In an example in FIG. 12D, a state in which the contents of processing such as “input” and “real scale display” and the evidence No. of “3” are output is illustrated. Subsequently, fields such as “evidence type” and “workflow step No.” are extracted from the extracted “evidence No.” and the evidence data table 310 (S100304, S100305), and are output to an evidence pattern table 1230 stored in the medical information database 105 illustrated in FIG. 12C (S100310). In an example illustrated in FIG. 12C, since the evidence No. such as “3” are extracted in S100303, a state in which evidence types including “laboratory data graph” and workflow No. such as “4” are output is illustrated. Subsequently, fields such as “programmed department of execution”, “parent workflow step No.”, and “child workflow step No.” are extracted from the extracted “workflow step No.” and the workflow step information table 210 (S100306, S100307). In an example of a workflow step pattern table 1220 in FIG. 12B, since the workflow step Nos. including “4” are extracted in S100304 and S100305, a state in which the programmed departments of execution such as “radiology diagnosis and treatment department”, the parent workflow step Nos. such as “3”, and the child workflow step Nos. such as “5” are output is illustrated. Subsequently, from the extracted “parent workflow step No.” and the workflow step information table 210, the fields such as “programmed department of execution” is extracted as the programmed parent department of execution (S100308). In the example illustrated in FIG. 12B, since the parent workflow step Nos. such as “3” are extracted in S100307, a state in which the programmed parent departments of execution such as “radiological technology department” are output is illustrated. In addition, from the extracted “child workflow step No.” and the workflow step information table 210, the fields such as “programmed department of execution” are extracted as a programmed child department of execution (S100309). In the example illustrated in FIG. 12B, since the child workflow step Nos. such as “5” are extracted in S100307, a state in which the programmed child departments of execution such as “internal medicine department” are output is illustrated. The “programmed department of execution”, the “programmed parent department of execution”, and the “programmed child department of execution” extracted in this manner are output to the workflow step pattern table 1220 stored in the medical information database (S100310). In this configuration, the respective tables as illustrated in FIG. 12B to FIG. 12D are created in the medical information database 105.

Subsequently, the clinical evaluation calculating means 1604 will be described in detail. The clinical indicator corresponds to a numerical value obtained by measuring the quality of medical treatment, and includes the No. of days of hospitalization, the rate of rehospitalization, and the rate of achievement of blood glucose control. Here, a flow of processing performed by the clinical evaluation calculating means 1604 will be described with reference to S1004 and S1005 in FIG. 10. First of all, in S1004, a clinical indicator for each of the workflows is calculated from the medical information database 105, the hospital information system, or the like. A state of calculation is illustrated in a clinical indicator table 1210 in FIG. 12A. The clinical indicator table 1210 is stored in the medical information database 105. A method of calculating the clinical indicator is described in detail in NPL 1. The clinical indicators may be calculated by the clinical evaluation calculating means 1604, or those calculated in advance and accumulated in the hospital information system such as electronic health record may be extracted. Subsequently, in S1005, the workflows are sorted in the descending order from a workflow in which a calculated clinical indictor is low to a workflow in which the same is high, and the sorted ranks are calculated as clinical evaluation values C(p, i) (where p is a patient ID, and i is a clinical indicator identifier for identifying the clinical indicator) with respect to the respective workflows. The reason why the sorted record is used is because various clinical indicators are used when evaluating the workflow steps and hence the evaluation is avoided from being related to the distribution of the values of the clinical indicators. Here, the values calculated in S1004 and the values normalized for the respective clinical indicators may be used as C(p, i) without performing S1005.

Subsequently, a first operation example of the workflow step evaluating means 1605 will be described in detail with reference to S1006 to S1008 in FIG. 10. First of all, in S1006, a flag F(p, s) indicating whether or not a workflow step pattern is executed from the table created in the data extracting means 1602 and the workflow step classifying means 1603 is calculated for each of workflow steps. Subsequently, in S1007, the number of times of execution of the workflow step pattern s N(s) is calculated. Finally, in S1008, the sum of the clinical evaluation values C(p, i) of the respective workflows executed in the workflow step s is calculated by using F(p, s), and the value obtained by dividing the calculated sum by the number of times of execution N(s) is calculated as a workflow step evaluation value. Accordingly, calculation of the respective workflow step evaluation values is enabled.

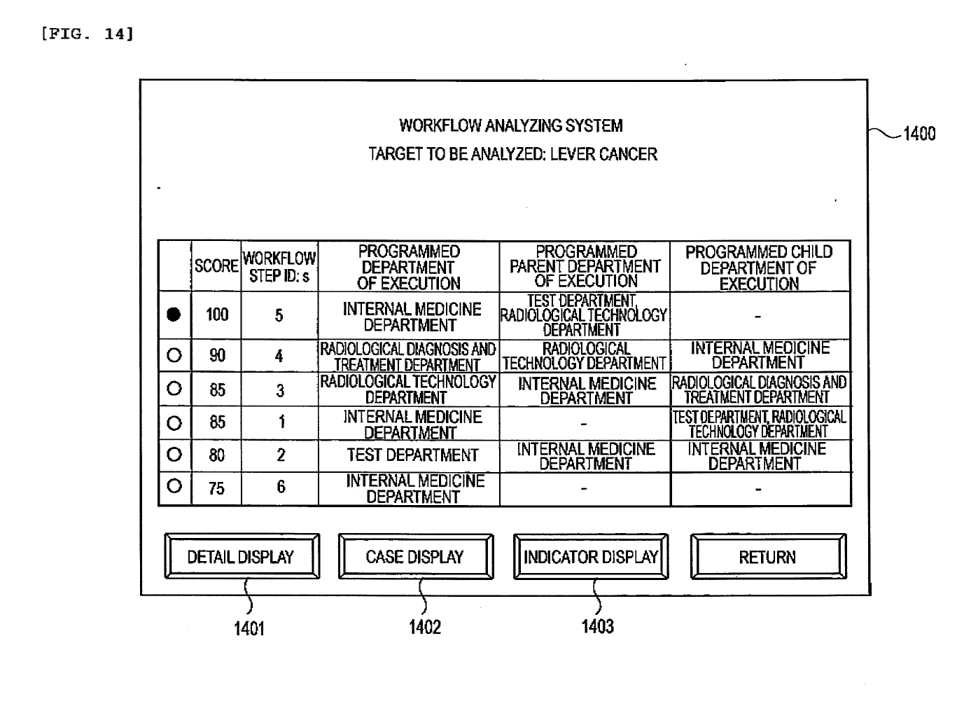

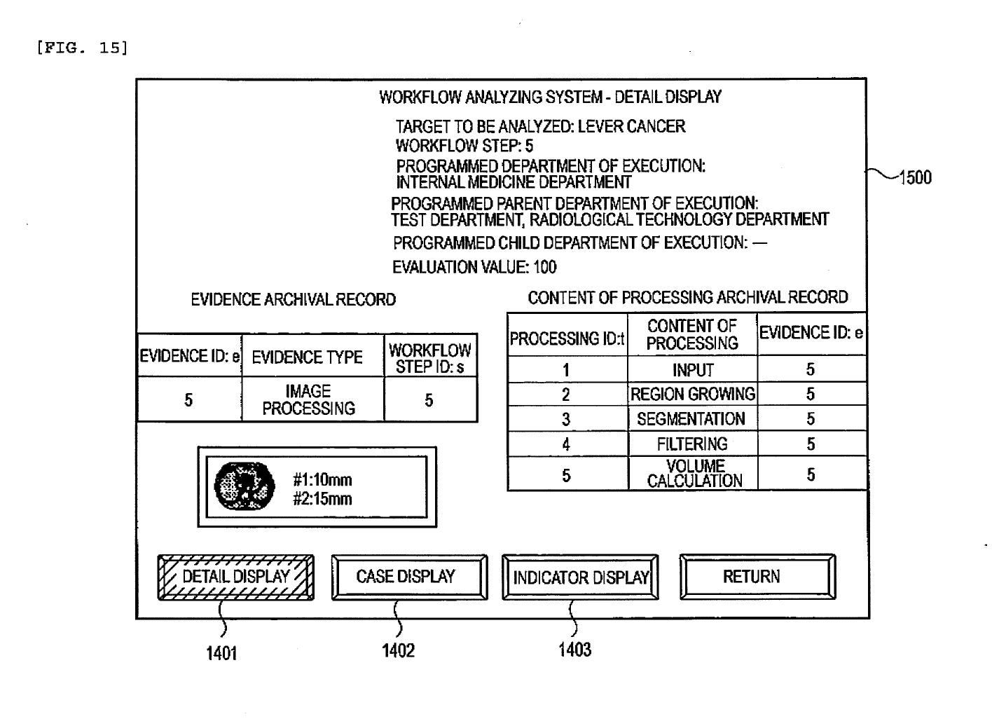

Subsequently, a screen for presenting a workflow step pattern configured by the display means 1606 and an evaluation value thereof will be described with reference to FIG. 13 to FIG. 17. FIG. 13 illustrates a disease selection screen 1300 showing a state of accepting a disease to be analyzed selected by a user. This selection is accepted by the disease selection means 1601. In this example, a state in which the user selects lever cancer (RFA) is illustrated, and when an analysis start button 1301 is pressed downward, a workflow step evaluation value screen 1400 as illustrated in FIG. 14 is displayed, and the workflow step evaluation value is presented. In this example, a state in which the evaluation value of the workflow step pattern in which the programmed department of execution is the internal medicine department and the programmed parent department of execution is the test department and the radiological technology department is as high as 100 is observed. Therefore, when the workflow step pattern having a high evaluation value is selected and a detail display button 1401 at a lower left of the screen is pressed downward, a workflow step evaluation value detail screen 1500 as illustrated in FIG. 15 is displayed, and detailed information on the selected workflow step pattern is presented. In this example, the image processing is performed in the selected workflow step pattern, and specifically, a state in which performing screen inputting, region growing, segmentation, filtering, and volume calculating processing are performed is observed. Here, the values of C(p, i) and F(p, s) are calculated as a function of p, which is a patient ID. However, it may be a function of workflow No. 202.

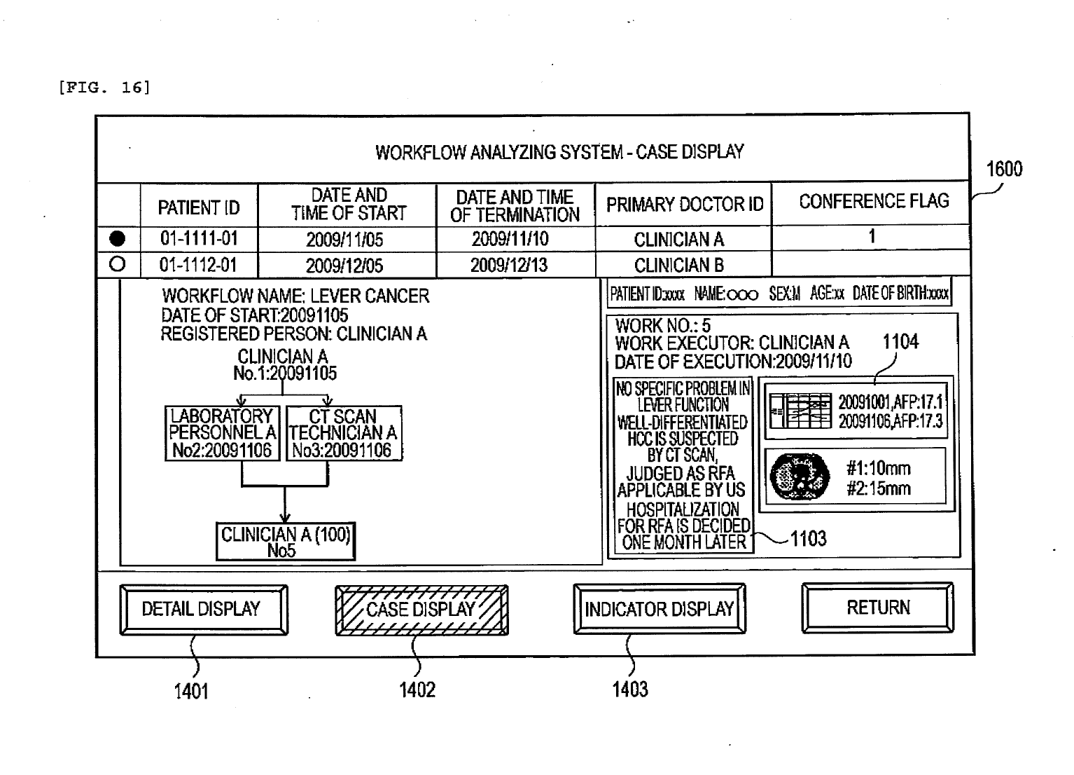

Subsequently, when a case display button 1402 is pressed downward, as illustrated in FIG. 16, a detailed case where the selected workflow step pattern is executed is presented on a workflow step evaluation value case display screen 1600. In this example, a case of the workflow step pattern executed for the patient ID: 01-1111-01 is displayed, and detail of a medical act by the clinician A is displayed. Subsequently, when an indicator display button 1403 is pressed downward, as illustrated in FIG. 17, a clinical indicator value of the workflow of the selected workflow step pattern is executed is presented on an indicator display screen 1700. With this screen, a clinical indicator which is a foundation of high workflow step evaluation value may be observed.

With these procedures, the evaluation of the workflow step, which is a detailed execution process of the workflow, which is a series of medical-related work of the medical practitioner, may be supported. Furthermore, since the workflow step may be evaluated, improvement of the workflow such as elimination of useless test may be supported by keeping the personnel informed about the workflow step whose workflow step evaluation value is high and, on the other hand, reviewing the workflow step whose workflow step evaluation value is low.

<Analysis Example 2 of Medical Care Process>

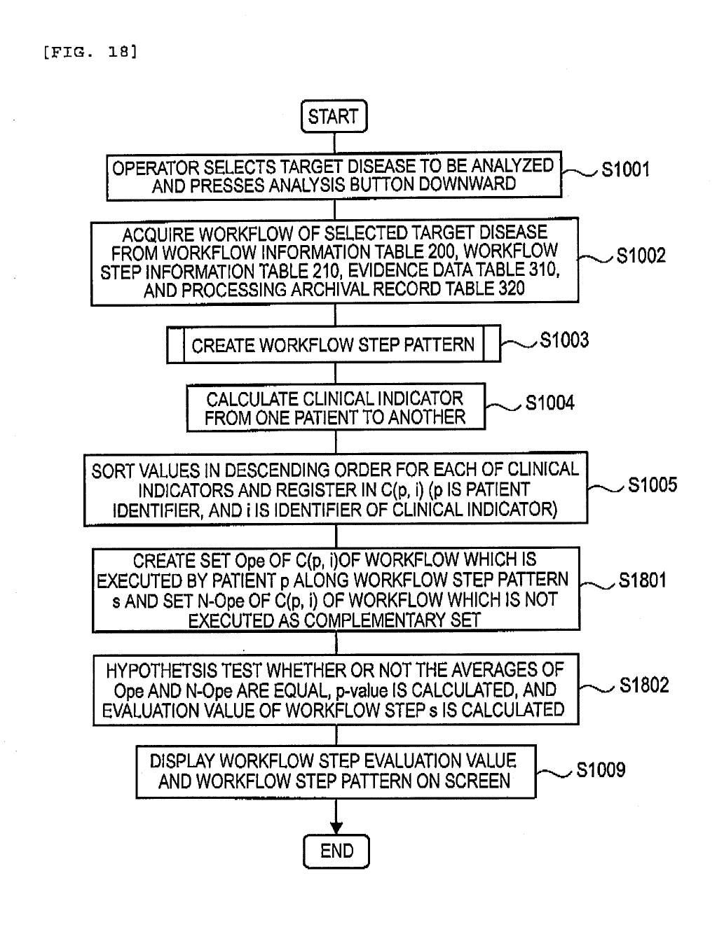

A second operation example of the workflow step evaluating means 1605 will be described. FIG. 18 illustrates an example of a second flowchart showing the outline of the operation of the process analyzing means 160. The difference from the flowchart in FIG. 10 is S1801 and S1802 which are processing relating to the workflow step evaluating means 1605. The workflow step evaluating means 1605 in FIG. 10 is calculated on the basis of the clinical evaluation value of the workflow which is executed along the workflow step pattern. In contrast, the workflow step evaluating means 1605 illustrated in FIG. 18 is different in that a clinical evaluation value of a workflow which is executed along the workflow step pattern and a clinical evaluation value of a workflow which is not executed along the workflow step pattern are calculated respectively and compared.

S1801 and S1802 in FIG. 18 will be described in detail. First of all, in S1801, a set Ope of C(p, i) of a workflow executed by a patient ID: p along the workflow step pattern s and a set N-Ope of C(p, i) of a workflow, which is not executed as a complementary set are created from the workflow step pattern created in S1003 and C(p, i) created in S1005. Subsequently, in S1802, a hypothesis test for checking whether or not the average of C(p, i) in Ope and N-Ope are equal is performed to calculate a significance probability p-value, and an evaluation value of the workflow step pattern S is calculated. In the hypothesis test, at test which is generally used may be applied. In order to calculate the evaluation value of the workflow step pattern s, the p-value may be used as an evaluation value or may be calculated by Expression 1.

$$\begin{matrix}[ Expression 1 ] & \\ [ Expression 1 ] & \\ \{ \begin{matrix}AVG ( 1 - [ p - value ] ) & \begin{matrix}Average value of indicator \\ evaluation value of Ope > average \\ value of indicator evaluation \\ value of N - OPE\end{matrix} \\ AVG ( [ p - value ] - 1 ) & Otherwise\end{matrix} & Expression ( 1 )\end{matrix}$$

The AVG is a value obtained by calculating p-value for each of the clinical indicator identifiers i that identify the clinical indicators, and calculating an average. In Expression 1, the higher the indicator evaluation value of the workflow in Ope, the higher the evaluation value of the workflow step pattern s becomes.

In this manner, by using the clinical evaluation value of the workflow which is not executed along the workflow step pattern of the object to be evaluated for calculation, whether the indicator evaluation value of the workflow is significantly increased by the execution of the focused workflow step may be evaluated by using the hypothesis test. In other words, evaluation based further on the statistical adequacy is enabled, and the higher accuracy is achieved. In addition, by presenting the p-value as a foundation on the workflow step evaluation value detail screen 1500 as illustrated in FIG. 15 together, the adequacy of the evaluation value of the workflow step may also be confirmed.

In this example, although the workflow step having a small p-value and affecting the clinical indicator is extracted, the item having a large p-value may be considered to be a workflow step which does not affect the clinical indicator. In other words, it may be considered to be an unnecessary workflow step. In this manner, by presenting the useless workflow step having a large p-value, improvement to a low-cost and high-quality workflow is enabled.

<Analysis Example 3 of Medical Care Process>

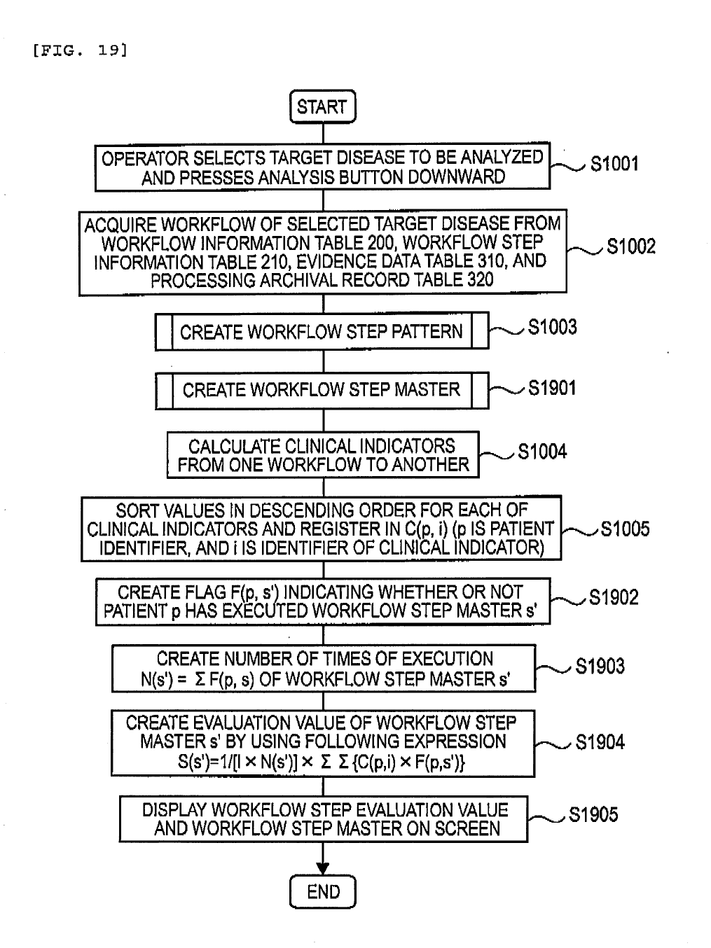

A second operation example of the workflow step classifying means 1603 will be described. FIG. 19 illustrates an example of a third flowchart showing the outline of the operation of the process analyzing means 160. The difference from the flowchart in FIG. 10 is that S1901 as a processing relating to the workflow step classifying means 1603 is added after S1003. In association with the addition of S1901, a point that the processing relating to the workflow step evaluating means 1605 is changed from S1006 to S1008 to S1902 to S1905 is also different.

Hereinafter, a detailed example of operation of S1901 will be described. First of all, an object of S1901 in this example will be described. The workflow step classifying means 1603 illustrated in FIG. 10 extracts the common medical-related work from the accumulated data and creates a workflow step pattern. However, there may be a case where a different workflow step pattern is created due to a small difference of the processing archival record in association with a scale conversion or an erroneous operation. Therefore, integration of workflow step patterns different due to small differences into one group is considered. This group is referred to as a workflow step master hereinafter. In order to create this workflow step master, small differences are defined and a workflow step patterns are integrated. Here, the small differences are defined as “processing archival record that does not affect the clinical indicators”.

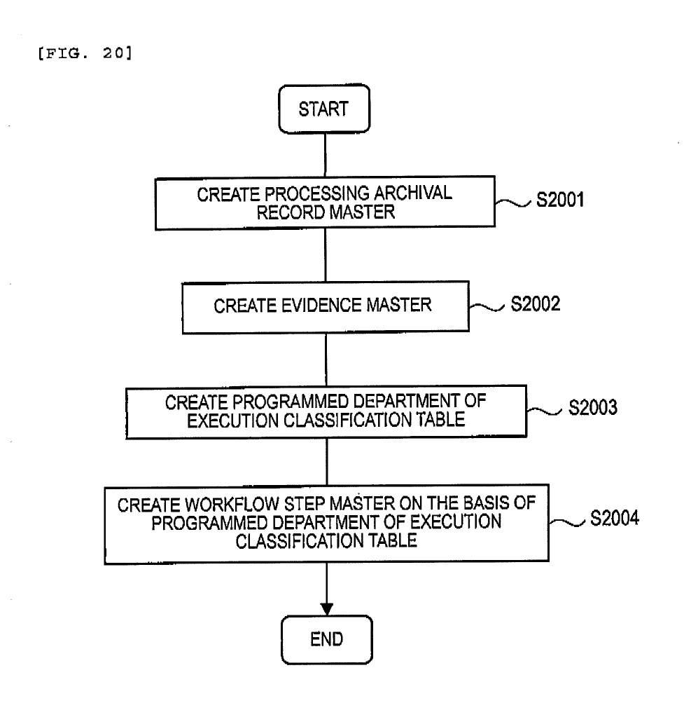

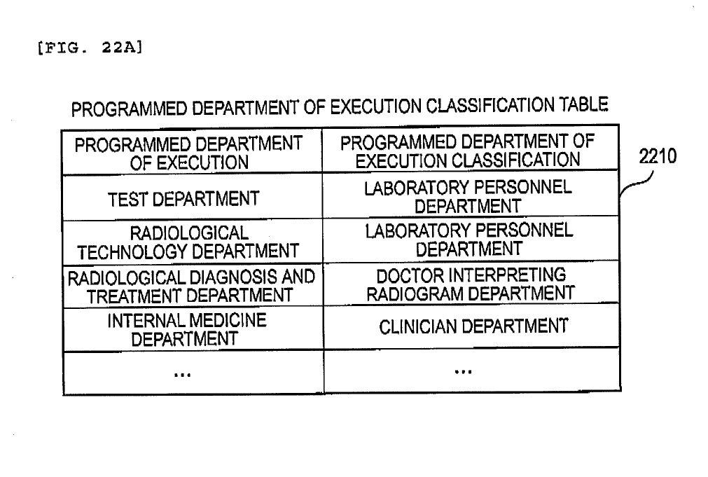

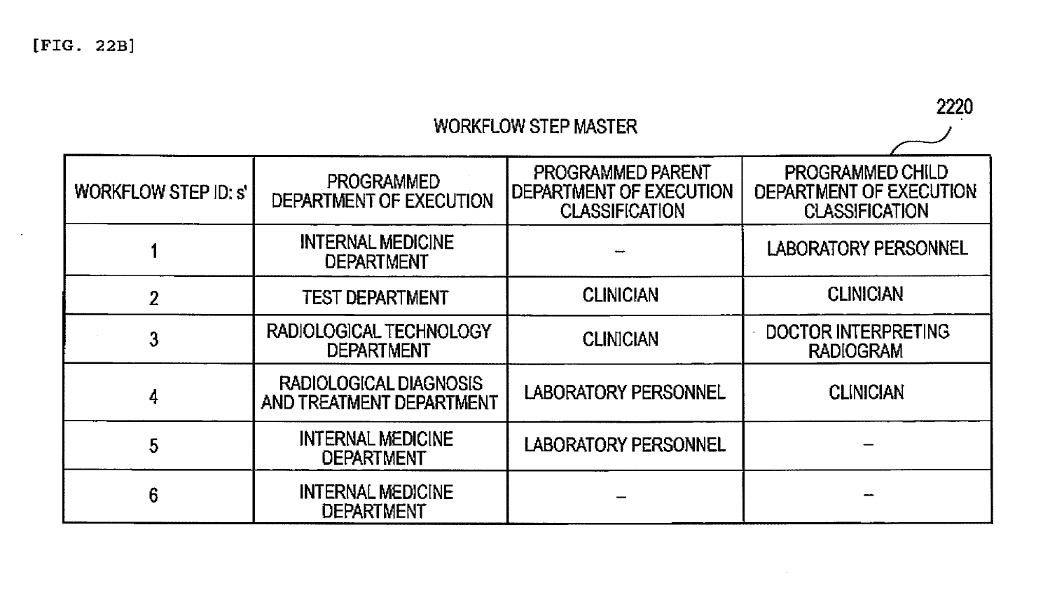

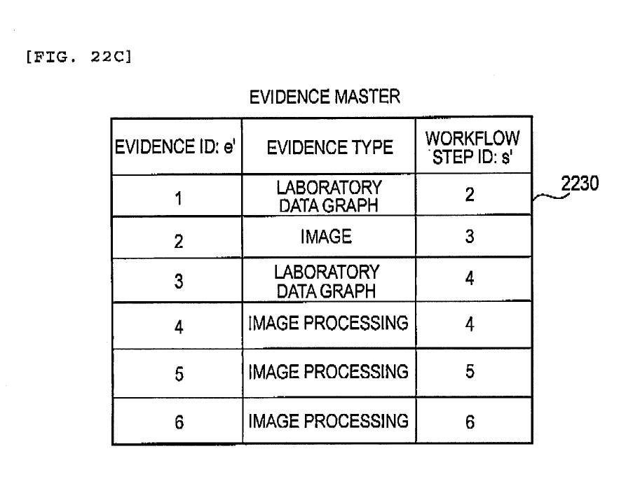

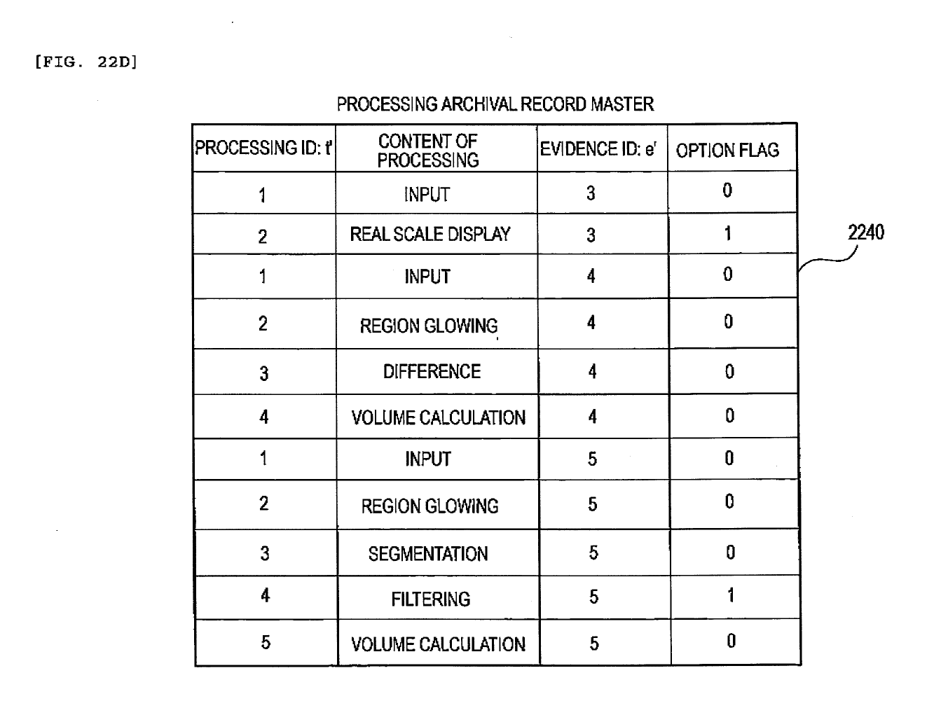

FIG. 20 is an example of a second flowchart illustrating an operation of the workflow step classifying means 1603 and is a detailed flowchart of S1901 in FIG. 19. In the example of the second flowchart, processing to be performed on the workflow step pattern obtained in the processing illustrated in FIG. 11 is shown. First of all, in S19011, small differences in the processing archival record are integrated to create a processing archival record master table 2240 illustrated in FIG. 22D.

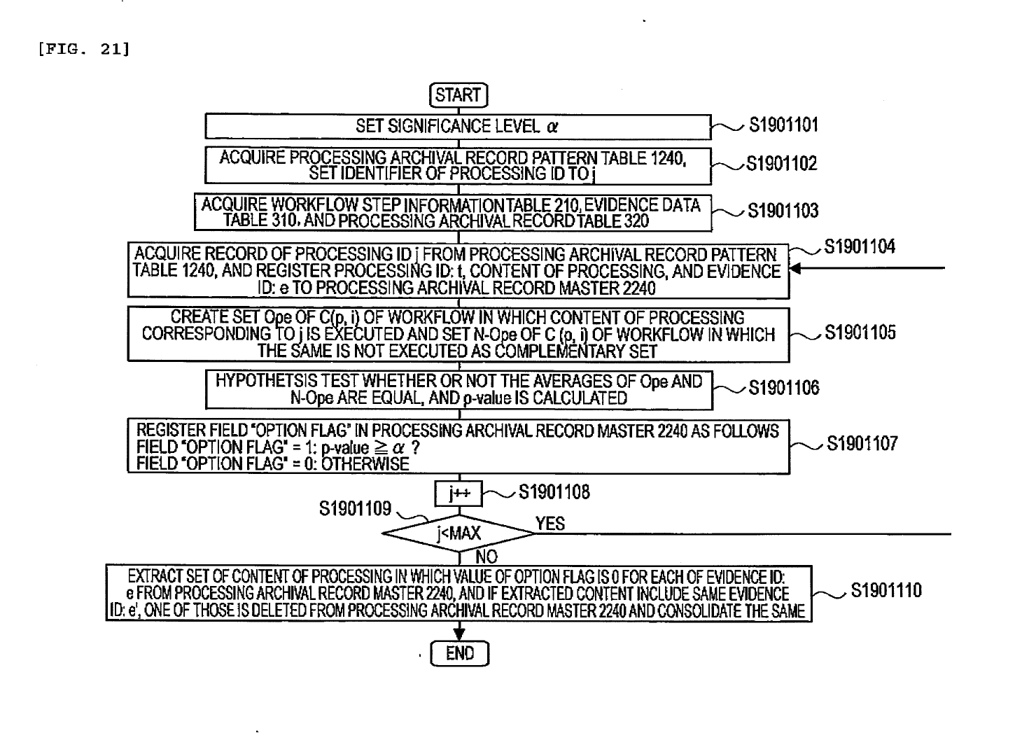

Here, the processing of S19011 will be described in detail. FIG. 21 illustrates a detailed flowchart of S19011 in FIG. 20. First of all, a significance level α is set (S1901101). The significance level α may be set in advance, or means configured to accept the significance level α may be provided in the disease selection means 1601. Subsequently, the processing archival record pattern table 1240 stored in the medical information database 105 illustrated in FIG. 12D is acquired to set the identifier j of the processing ID (S1901102). Subsequently, the following processing is performed on the respective records (S1901103 to S1901108). First of all, the record of the identifier j of the processing ID is acquired from the processing archival record pattern table 1240 (S1901103) to register the processing ID, a content of the processing, and an evidence ID of the identifier j in the processing archival record master table 2240 (S1901104). Subsequently, the set Ope of the clinical evaluation value C(p, i) of the workflow in which the content of the currently focused processing is executed and the set N-Ope of the clinical evaluation value C(p, i) of the workflow which is not executed are created in the same processing as that in S1801 (S1901105). Subsequently, a hypothesis test for checking whether the averages of the Ope and the N-Ope are equal is performed, and p-value is calculated (S1901106). Subsequently, a field “option flag” is registered in the processing archival record master table 2240 in accordance with the p-value (S1901107). The field “option flag” is a flag which indicates whether or not there is a small difference in processing archival record, and provides “1” when the difference is small and “0” when the difference is large. Accordingly, if the p-value is larger than the significance level α, the focused processing archival record pattern does not affect on the clinical indicator, so that the option flag is provided with “1”. In contrast, if the p-value is smaller than the significance level α, the focused processing archival record pattern affects on the clinical indicator, so that the option flag is provided with “0”. In this manner, the set of the content of the processing in which the value of the option flag is “0” is extracted for each of the evidence IDs from the processing archival record master table 2240 registered in this manner and, if evidence IDs whose extracted contents are the same exist, one of those is deleted from the processing archival record master table 2240 and integrated (S1901110).

After the creation of the processing archival record master table 2240 in this manner, if an evidence archival record is to be integrated as a result of S1901110, an evidence master table 2230 illustrated in FIG. 22C is created in the medical information database 105 by the integration (S19012). Also, a programmed department of execution classification table 2210 in which the programmed department of execution illustrated in FIG. 22A is stratified is created in the medical information database 105 (S2003). This is a processing for integrating the test department and the radiological technology department or the like as a laboratory personnel department. This processing may be set by the user in advance, or may integrate the programmed department of execution appearing at the same time as a parent and child identifier. In S2004, when the workflow step archival record is integrated as a result of S2109, a workflow step master table 2220 is created in the medical information database 105 illustrated in FIG. 22B by the integration (S2004).

Accordingly, an important archival record affecting the clinical indicator may be focused for analysis, and hence the workflow step performed with improved data processing may be evaluated, so that an effect of further increase in efficiency of analysis is achieved.

<Analysis Example 4 of Medical Care Process>

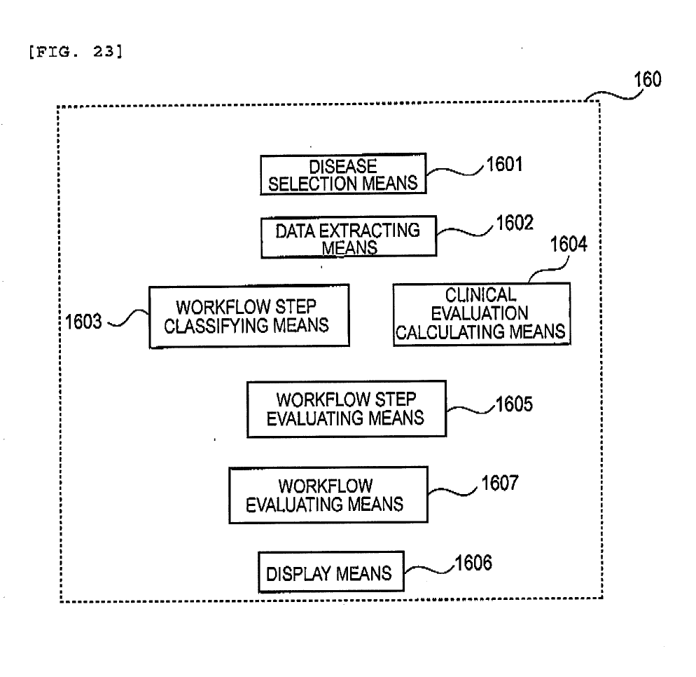

FIG. 23 illustrates an example of second configuration means of the process analyzing means 160 of the present invention. In particular, workflow evaluating means 1607 is newly added to a configuration drawing illustrated in FIG. 9 for the purpose of the evaluation of the workflow. The workflow evaluating means 1607 realizes various processing by deployment and activation of predetermined programs in the CPU 114 and the memory 112 or the like illustrated in FIG. 1A.

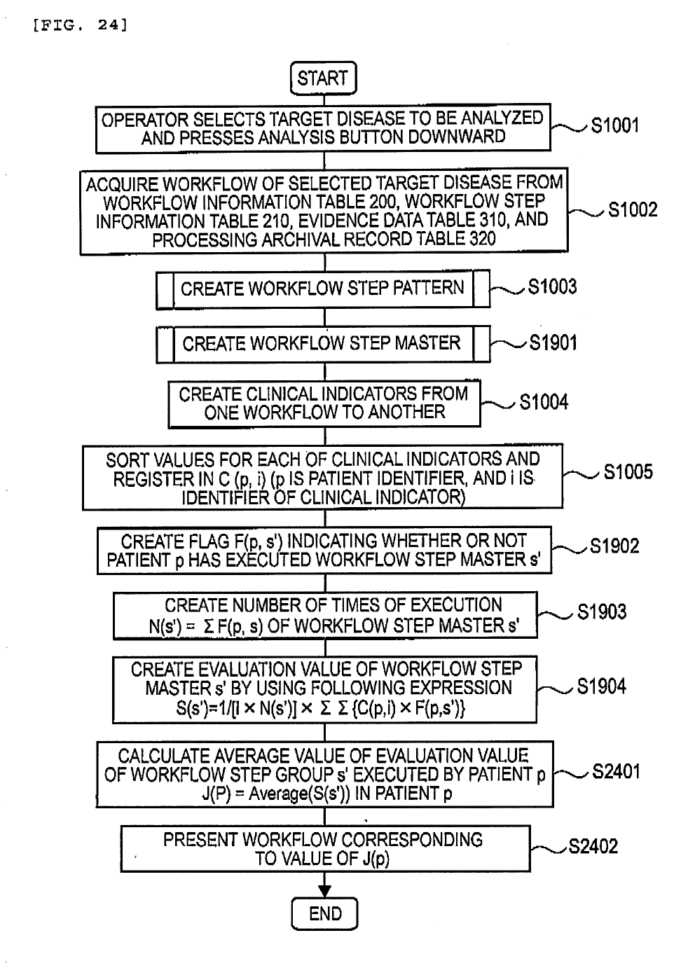

FIG. 24 is a fourth flowchart example illustrating an outline of the operation of the process analyzing means 160, and the difference from the flowchart in FIG. 19 is that S2401, which is a processing relating to the workflow evaluating means 1607 is added. In association with it, a point that the processing relating to the display means 1606 is changed from A1905 to S2402 is also different.

Hereinafter, S2401 and S2402 will be described. In S2401, an average value of the workflow step evaluation values is calculated for each of the patient IDs: p for calculating the workflow evaluation value, and the obtained value is used as a workflow evaluation value. Here, the workflow evaluation value is calculated on the basis of the workflow step master table 2220. However, the workflow evaluation value may be calculated on the basis of the workflow step evaluation value calculated for each of the workflow step patterns calculated in FIG. 10 or in FIG. 17.

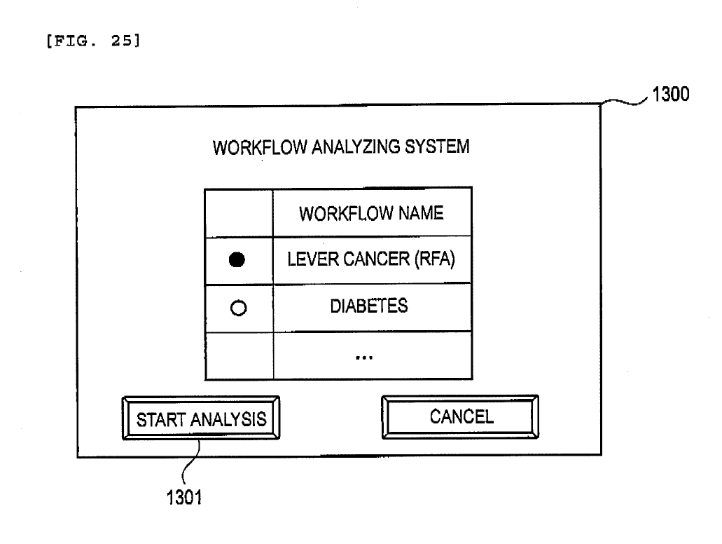

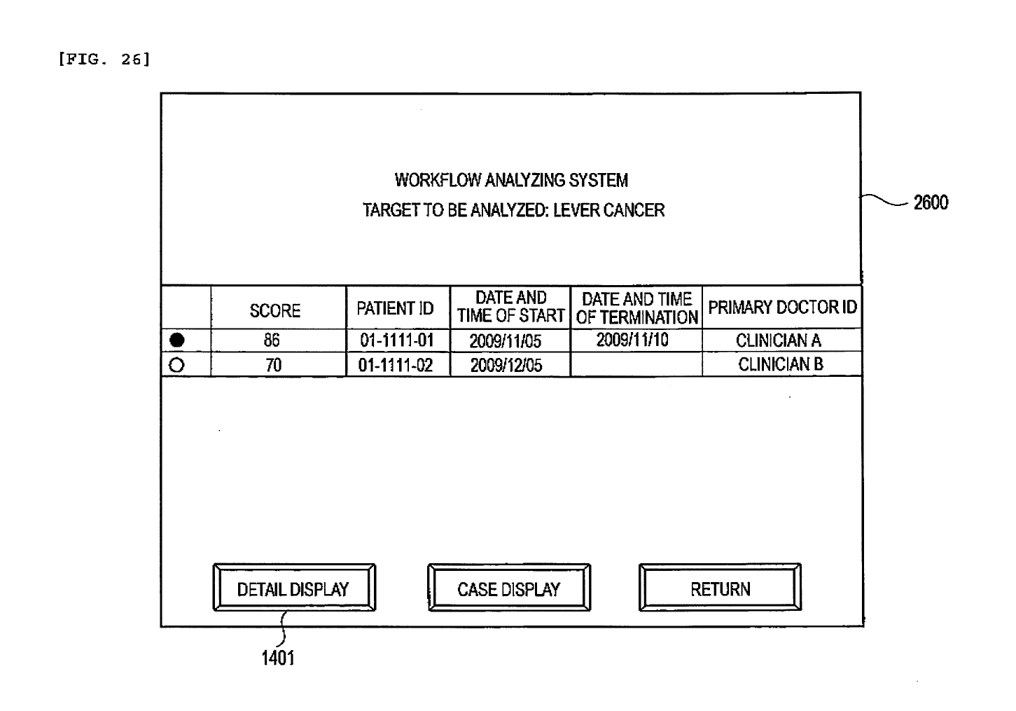

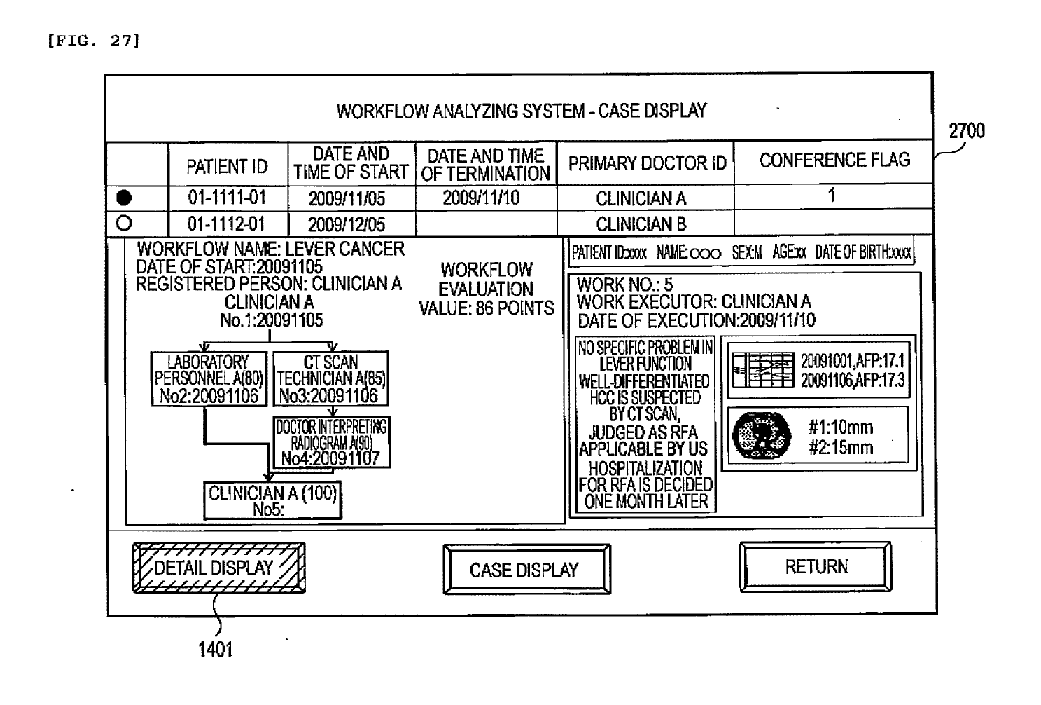

In S2402, the workflow is presented on a screen together with the workflow evaluation value. Referring to FIG. 25 to FIG. 27, the screen for presenting the workflow together with the workflow evaluation value will be described. In FIG. 25, in the same manner as FIG. 13, a state in which the user selects a disease to be analyzed, which is a screen corresponding to the disease selection means 1601, is illustrated. In this example, a state in which the user selects lever cancer (RFA) is illustrated, and when the analysis start button 1301 is pressed downward, a workflow evaluation value screen 2600 as illustrated in FIG. 26 is displayed, and the workflow evaluation value is presented. FIG. 26 resembles to FIG. 14, but is different from FIG. 14 in that the workflow step evaluation values are presented for the respective workflow step patterns in FIG. 14, while the workflow evaluation values are presented from one patient to another in FIG. 26. In this example, a state in which the workflow evaluation value of the patient ID: 01-1111-01 is high is observed. When the workflow is selected and the detail display button 1401 at a lower left of the screen is pressed downward, a workflow evaluation value case display screen 2700 as illustrated in FIG. 27 is displayed, and detailed information of the selected workflow is presented. In this example, a workflow, which is a flow of the workflow step, is presented, and a workflow step evaluation value is also presented in a box indicating the respective workflow steps. For example, a state in which the workflow step evaluation value of the workflow step No. 5 executed by a clinician is 100 may be observed and, specifically important workflow steps may be confirmed.

Accordingly, evaluation of the workflow is achieved by statistically processing the clinical evaluation values of the individual workflows. The clinical evaluation values of the individual workflows are calculated on the basis of the individual identical clinical indicators. However, since the workflow evaluation value is obtained by processing the clinical evaluation value statistically, a workflow which has a salutary effect on the clinical indicator may be statistically confirmed without depending on the initial state of the individual patients, for example. In addition, as illustrated in the right portion of the screen in FIG. 27, the processing archival record and the process of judgment of the medical practitioner may be presented in correspondence to each other, so that the background and a thinking process of the medical-related work may be grasped. In other words, the analysis of the workflow is achieved while confirming the process of the detailed medical-related work such as diagnosis or therapy or implicit knowledge.

<Analysis Example 5 of Medical Care Process>

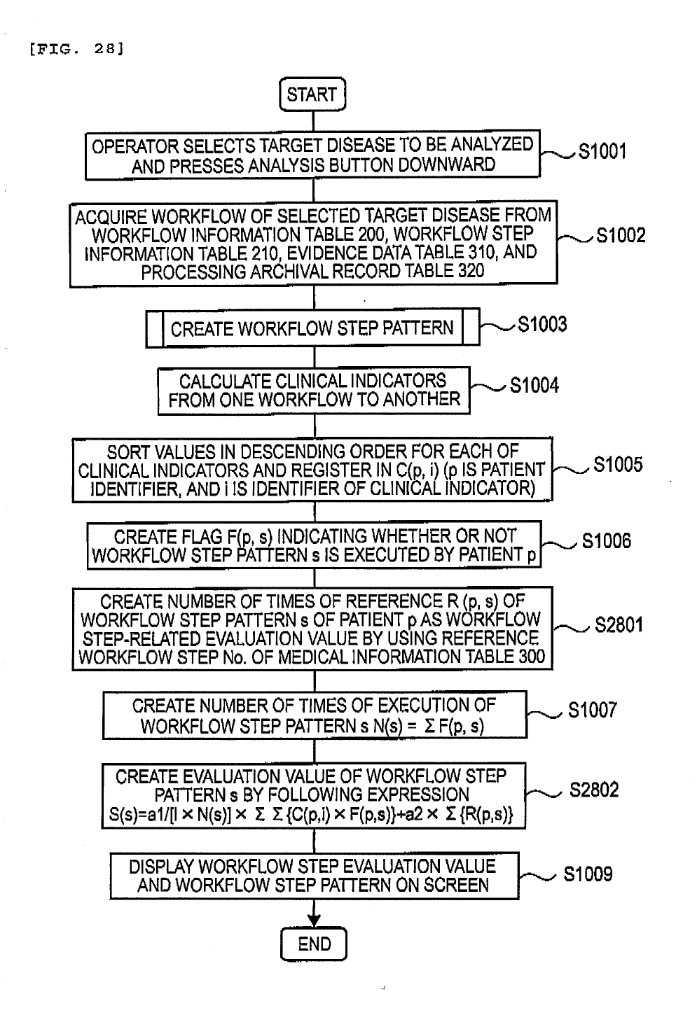

FIG. 28 illustrates an example of a fifth flowchart showing the outline of the operation of the process analyzing means 160. This example is specifically characterized in that a workflow step-related evaluation value is calculated by using the reference workflow step No. 306 to enhance the accuracy of the workflow step evaluation. The workflow step-related evaluation value is a value obtained by converting the number of times of being referred to in other workflow steps into an indicator. The difference from the flowchart in FIG. 10 is S2801 and S2802 which are processing relating to the workflow step evaluating means 1605.

Hereinafter, a detailed example of operation of S2801 and S2802 will be described. In S2801, the reference workflow step No. 306 of the medical information table 300 is acquired, and the number of times of reference R(p, s′) to from the workflow step s′ of the patient p to the workflow step pattern is created as a workflow step-related evaluation value. In S2802, a sum of an evaluation value calculated in S1008 in FIG. 10 assigned with a weight by a predetermined constant a1 and a value calculated on the basis of the workflow step-related evaluation value assigned with a weight by a predetermined constant a2 is calculated as an evaluation value of the workflow step pattern. In this flowchart, the processing relating to the workflow step evaluating means 1605 is changed on the basis of FIG. 10. However, it is also possible to calculate a workflow step pattern added with the workflow step-related evaluation value or an evaluation value of the record in the workflow step master table 2220 on the basis of FIGS. 18, 19, and 24. Although the number of times of reference R is a function of “p” as the patient ID, it may be used as a function of the workflow No. 202 for calculation.

Accordingly, since the reference of the workflow step as a visualized implicit knowledge is taken into consideration at the time of the workflow step evaluation in addition to the evaluation on the basis of the clinical indicator, the workflow step considered to be important in the judgment of the medical-related work which has been the implicit knowledge may be extracted and evaluated.

<Analysis Example 6 of Medical Care Process>

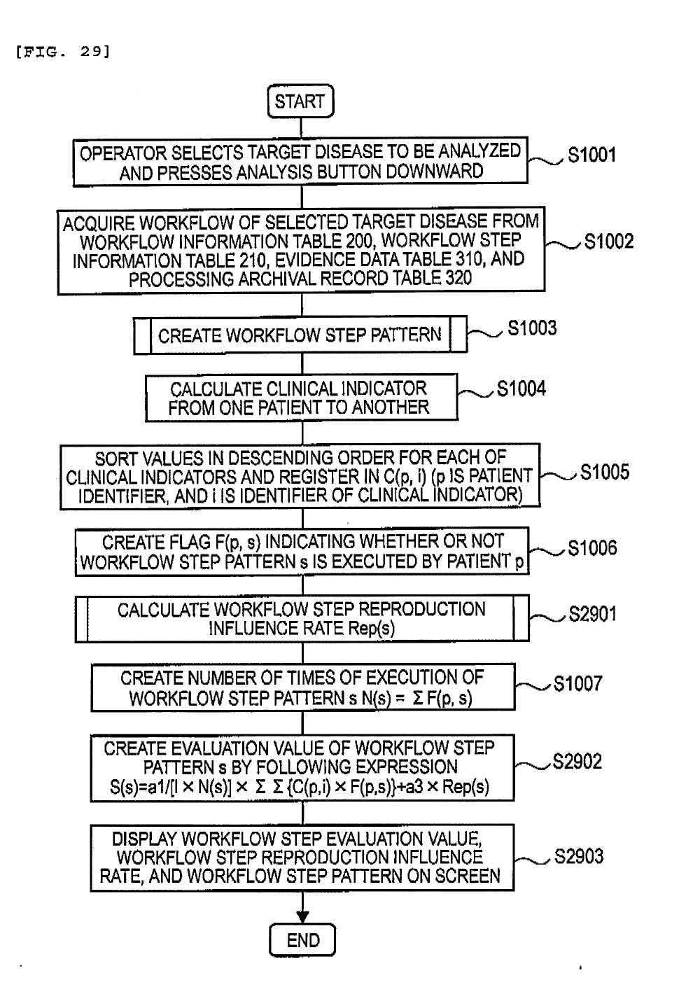

FIG. 29 illustrates an example of a sixth flowchart showing the outline of the operation of the process analyzing means 160. This example is specifically characterized in that information reproducing the processing archival record in the medical information referred to is taken into consideration, which is one of characteristics of the present system. In other words, if the clinical indicator is improved as a result of performing different processing after the reproduction of the processing archival record, the newly performed processing is regarded as an effective processing, and is accumulated as a new knowledge.

The difference of FIG. 29 from the flowcharts in FIG. 10 and FIG. 28 is S2901 and S2902 which are processing relating to the workflow step evaluating means 1605. In association with it, a point that the processing relating to the display means 1606 is changed from S1009 to S2903 is also different.

Hereinafter, the respective processing will be described in detail.

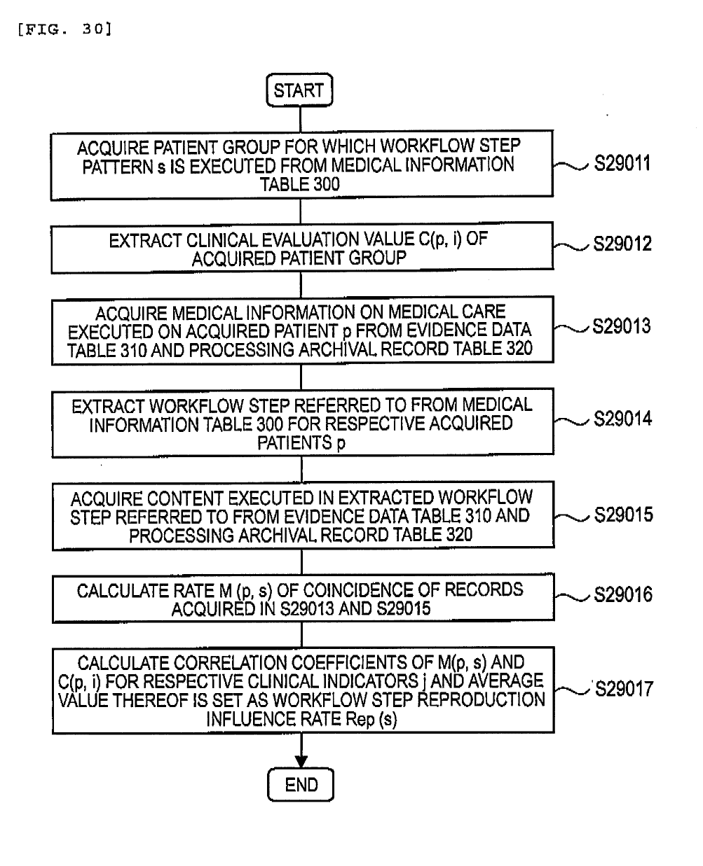

In S2901, a workflow step reproduction influence rate Rep(s) which indicates the degree of improvement of the clinical indicator as a result of performing different processing after the reproduction of the processing archival record is calculated. A detailed flow in S2901 is illustrated in FIG. 30. In the flow illustrated in FIG. 30, a rate of coincident of the processing archival records before the reproduction and after the reproduction and the clinical indicator are compared and converted into an indicator, which is used as the workflow step reproduction influence rate Rep (s). Specifically, first of all, a patient group having executed the workflow step pattern s is acquired from the medical information table 300 (S29011). Subsequently, a clinical evaluation value C(p, i) of the acquired patient group is extracted (S29012). Subsequently, the rate of coincidence of the processing archival records before the reproduction and after the reproduction is calculated in S29013 to S29016. In other words, the evidence type and the content of processing are acquired first from the evidence data table 310 and the processing archival record table 320 as information corresponding to the content executed for the acquired patients p (S29013). Subsequently, the reference workflow step No. 306 is extracted from the medical information table 300 for each of the acquired patients p (S29014). Subsequently, records associated with the evidence No. 311 executed in the extracted workflow step referred to and the processing No. 321 corresponding to the evidence No. thereof are acquired from the evidence data table 310 and the processing archival record table 320 (S29015). Finally, a rate M(p, s) of coincidence between the evidence type and the content of processing acquired in S29013 and S29015 are calculated from the extracted evidence data table 310 and the processing archival record table 320 (S29016). With respect to the rate of coincidence between the processing archival records before the reproduction and after the reproduction calculated in this manner and the clinical indicator, coefficients of correlation of a set of values M(p, s) and C(p, i) of the respective patients p are calculated for the respective clinical indicators i, an average value of the respective “i”s of the calculated coefficients of correlation is calculated, so that the workflow step reproduction influence rate Rep(s) is obtained (S29017). Here, the rate M and the clinical evaluation value C are functions of the patient p, however, they may be used as functions of the workflow No. 202 for calculation.

In S2902, a workflow step evaluation value considering the workflow step reproduction influence rate Rep(s) calculated in S2901. Specifically, a sum of an evaluation value calculated in S1008 in FIG. 10 assigned with a weight by the predetermined constant a1 and the workflow step reproduction influence rate Rep(s) assigned with a weight by a predetermined constant a3 is calculated as an evaluation value of the workflow step pattern. In this flowchart, the processing relating to the workflow step evaluating means 1605 is changed on the basis of FIG. 10. However, it is also possible to calculate the workflow step pattern added with the workflow step reproduction influence rate or an evaluation value of the workflow step master on the basis of FIGS. 18, 19, 24 and 28. Also, it is further possible to calculate the workflow step evaluation value by obtaining a sum with the workflow step-related evaluation value.

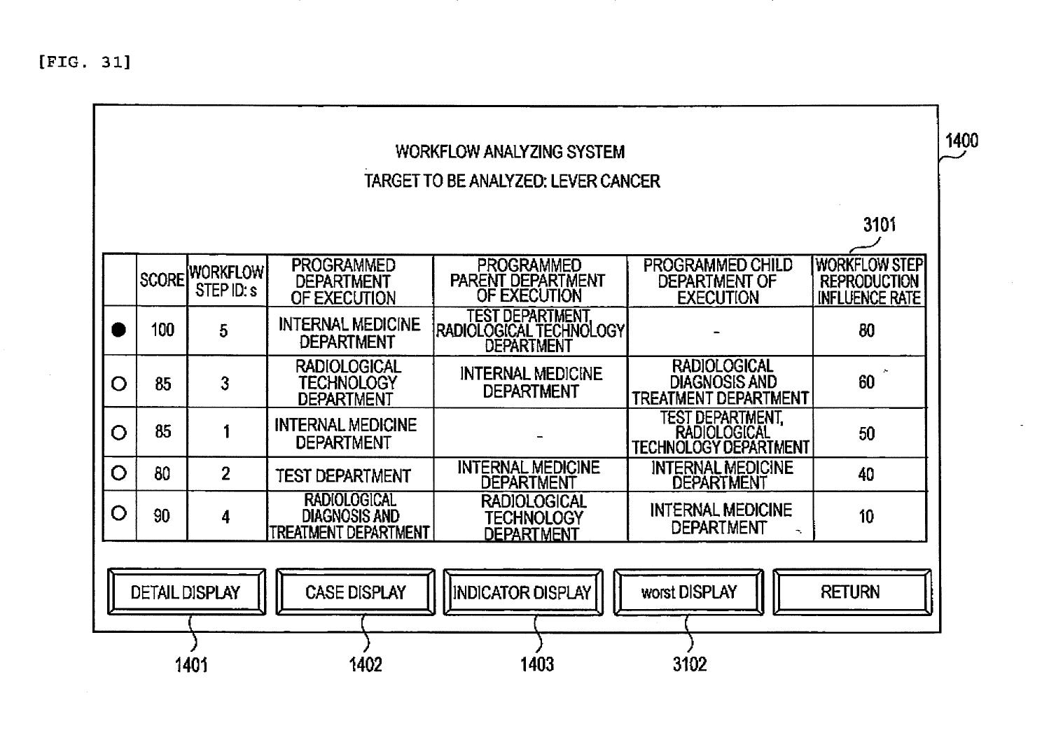

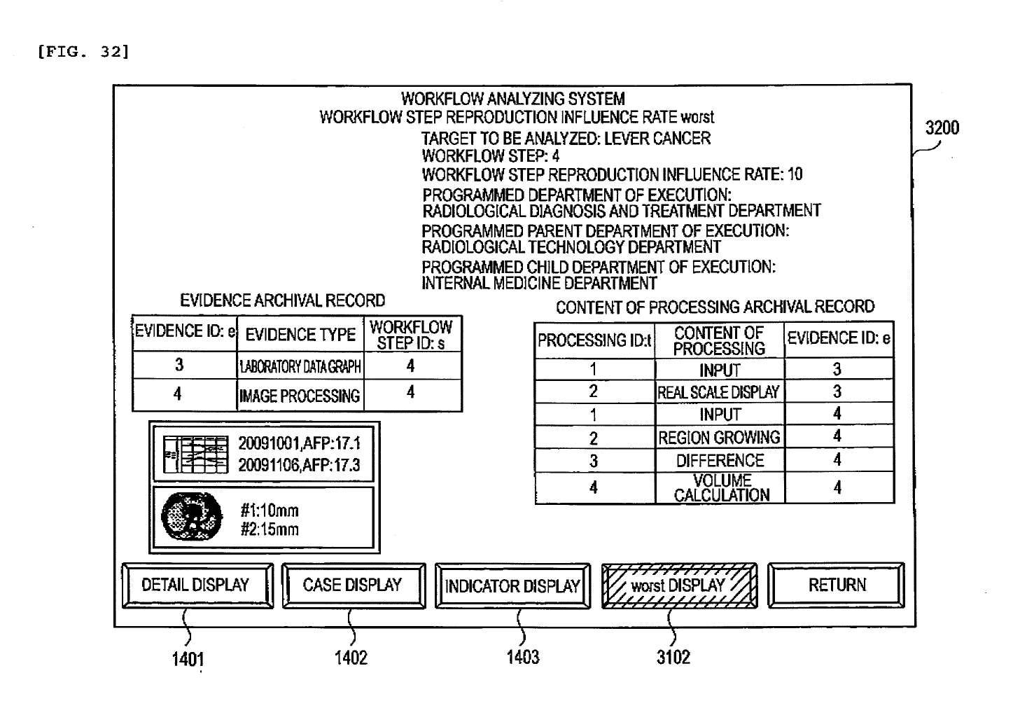

In S2902, the workflow step reproduction influence rate is also presented on the screen at the same time with the workflow step pattern. Referring to FIG. 31, a first screen configuration example will be described. In FIG. 31, the workflow step reproduction influence rate is displayed in a workflow step reproduction influence rate field 3101 together with the workflow step pattern and the workflow step evaluation value. Here, when a worst display button 3102 is pressed downward, a screen illustrated in FIG. 32 is displayed, and a workflow step pattern having the lowest workflow step reproduction influence rate is displayed on a workflow step reproduction influence rate worst screen 3200. When comparing with the workflow step pattern having a high workflow step evaluation value or the like, it is found that processing not included in the content of processing archival record in FIG. 32 is important processing, and the workflow steps to be improved may be confirmed.

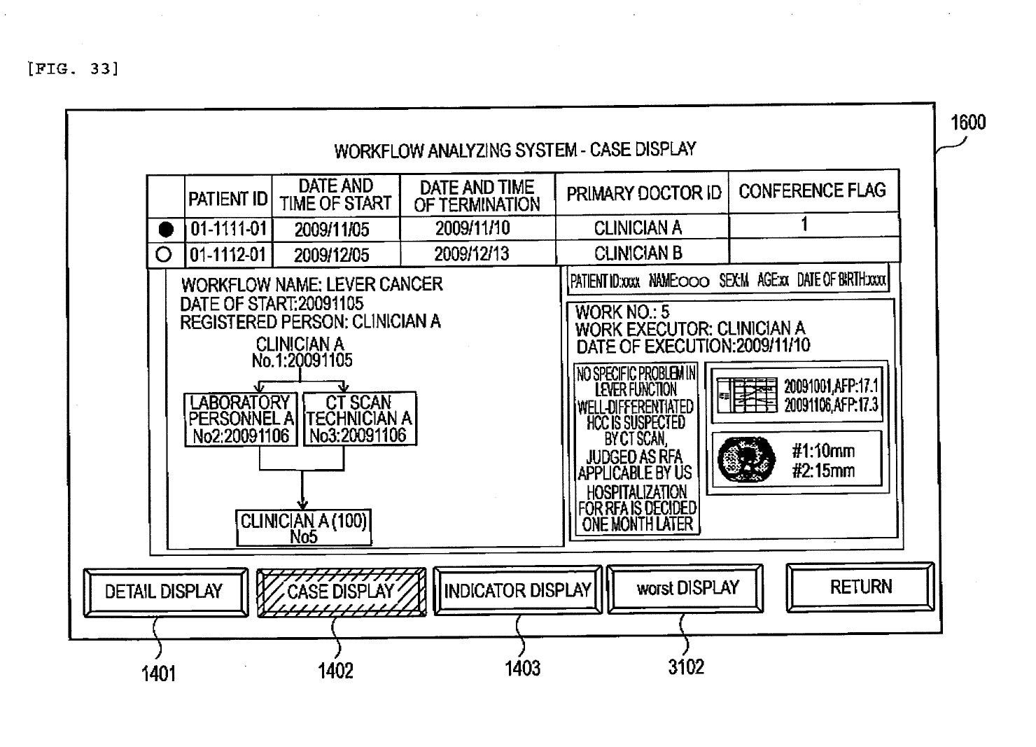

Subsequently, referring to FIG. 33, a second screen configuration example will be described. In this example, the screen is displayed by the case display button 1402 in FIG. 31 being pressed downward. The workflow executed along the selected workflow step pattern is displayed. Furthermore, information on the selected workflow is displayed on a lower part of the screen. In this example, a state in which a flow of a doctor interpreting radiogram whose workflow step reproduction influence rate Rep (s) exceeds a certain threshold value to a clinician is emphasized by a thick line is observed. This method of display allows control of width, color, or the like of the thick line according to the workflow step-related evaluation value. Accordingly, reference to the important workflow step may be visually grasped.

Accordingly, extraction of the workflow steps which improve the clinical indicator is enabled, and improvement of the workflow may be supported. In particular, since the workflow steps in which different types of processing are performed after the reproduction of the processing archival record are extracted, an optimal workflow step of the workflow steps which are not recognized as the optimal workflow step and are in try and select may be reviewed by using the reproduction influence rate. Since the workflow step pattern having a low workflow step evaluation value may be extracted easily as illustrated in FIG. 32, improvement of the workflow may be supported.

The present invention relates to a technology which may be used for a medical care support system treating laboratory data and image data of patients and remarks of medical practitioners, and to a technology for supporting analyses in a medical-related work performed by the medical practitioners.

### REFERENCE SIGNS LIST

101 medical care support system
102 electronic health record system
103 PACS
104 terminal
105 medical information database
106 medical information storage means
107 workflow input means
108 input information accepting means
109 medical information output means
111 interface
112 memory
113 memory device
114 CPU
115 evidence data processing means
116 laboratory information database
117 workflow step request input means
118 workflow output means
119 workflow terminating means
160 process analyzing means
200 workflow information table
201 patient ID field
202 workflow No. field
203 workflow name field
204 workflow start date and time field
205 workflow termination date and time field
206 primary doctor ID field
207 conference flag field
210 workflow step information table
211 patient ID field
212 workflow step No. field
213 programmed department of execution of workflow step 1D field
214 workflow step execution date and time field
215 workflow step executioner ID field
216 workflow step execution flag field
217 conference step flag field
218 workflow No. field
219 parent workflow step No. field
220 child workflow step No. field
300 medical information table
301 patient ID field
305 judgment sentence field
306 reference workflow step No. field
310 evidence data table
311 evidence No. field
312 evidence type field
313 evidence display icon field
314 evidence display text field
320 processing archival record table
321 processing No. field
322 processing content field
323 evidence number field
324 processing parameter field
330 input data table
331 evidence No. field
332 input data ID field
333 input data type field
400 laboratory data table
401 laboratory data ID field
402 patient ID field
403 laboratory result date and time field
404 item code field
405 value field
410 test item master table
411 item code field
412 item name field
413 unit name field
420 measured value table
421 measured value ID field
422 patient ID field
423 measured result date and time field
424 item code field
425 value field
430 measurement item master table
431 item code field
432 item name field
433 unit name field
434 date of registration field
440 image table
441 image ID field
442 patient ID field
443 image acquisition date field
444 item code field
445 image field
700 workflow step execution screen
701 workflow step selection area
702 login information display area
703 judgment sentence I/O area
704 evidence data display area
705 medical information registration button
706 medical care support button group
710 laboratory data graph display screen
800 image processing screen
1210 clinical indicator table
1220 workflow step pattern table
1230 evidence pattern table
1240 processing archival record pattern table
1300 disease selection screen
1301 analysis start button
1400 workflow step evaluation value screen
1401 detail display button
1402 case display button
1403 indicator display button
1500 workflow step evaluation value detail screen
1600 workflow step evaluation value case display screen
1601 disease selection means
1602 data extracting means
1603 workflow step classifying means
1604 clinical evaluation calculating means
1605 workflow step evaluating means
1606 display means
1607 workflow evaluating means
1700 indicator display screen
2210 programmed department of execution classification table
2220 workflow step master table
2230 evidence master table
2240 processing archival record master table
2600 workflow evaluation value screen
2700 workflow evaluation value case display screen
3101 workflow step reproduction influence rate field
3102 worst display button
3200 workflow step reproduction influence rate worst screen

1. A medical care support system comprising:
a medical information database in which a workflow information configured to identify a workflow as a flow of a series of medical care of a medical-related work, a workflow step item as information configured to identify a workflow step as a unit of medical-related work which constitutes the workflow, a disease identifier configured to identify a disease, a clinical indictor as an indicator of a quantitative evaluation of the quality of medical care are stored in association with each other;
a disease name input accepting unit configured to accept an input of a first disease identifier;
a workflow information extracting unit configured to extract a first workflow group including a plurality of items of workflow information associated with the first disease identifier whose input is accepted from the medical information database;
a workflow clinical evaluation value calculating unit configured to acquire the clinical indicators associated with the respective items of workflow information of the first workflow group respectively from the medical information database and calculate workflow clinical evaluation values, which are clinical evaluation values of the workflow with respect to the respective items of workflow information of the first workflow group on the basis of the acquired clinical indicators;
a workflow step evaluation value calculating unit configured to extract a first workflow step item included in the first workflow group from the medical information database respectively and calculate a workflow step evaluation value, which is an evaluation value of the first workflow step item on the basis of the first workflow group and a plurality of the calculated workflow clinical evaluation values; and
a screen output unit configured to output the first workflow step item and the calculated workflow step evaluation values to a screen.

2. The medical care support system according to claim 1, wherein
the workflow step evaluation value calculating unit
calculates a product of the number of the first workflow step items and the calculated workflow clinical evaluation value with respect to the respective items of workflow information of the first workflow group,
calculates a sum of products, which is a sum of the calculated products for the first workflow step items in the first workflow group, and
calculates a summation of the number of the first workflow step items in the first workflow group, and calculates a value obtained by dividing the sum of products by the summation as the workflow step evaluation value.

3. The medical care support system according to claim 1, wherein
the workflow step item includes a parent and child identifier for identifying a precedent or subsequent workflow step with respect to the workflow step, and the first workflow step item is a workflow step item identified by a first parent and child identifier.

4. The medical care support system according to claim 1, wherein
the workflow step evaluation value calculating unit extracts a workflow information set including the first workflow step item from the first workflow group as a workflow set to be executed, extracts a workflow information set in which the first workflow step item is not included from the first workflow group as a workflow set not to be executed, and calculates the first workflow step evaluation value on the basis of the respective workflow clinical evaluation values in the workflow set to be executed and the workflow set not to be executed.

5. The medical care support system according to claim 1, further comprising a workflow step classifying unit configured to extract workflow information including the first workflow step item as a workflow set to be executed from the first workflow group, extract a set of a workflow information not including the first workflow step item as a workflow set not to be executed from the first workflow group, calculate a significance probability by performing a hypothesis test on the basis of the respective workflow clinical evaluation values in the workflow set to be executed and the workflow set not to be executed, create a master identifier on the basis of the calculated significance probability, and store the master identifier in the medical information database with the master identifiers associated with the workflow step items, wherein
the workflow step evaluation value calculating unit extracts the first workflow step item included in the first workflow group from the medical information database on the basis of the master identifier.

6. The medical care support system according to claim 1, wherein
the workflow step item is stored in the medical information database in association with a patient identifier for identifying a patient as a subject of medical care in the workflow step, and
the workflow step evaluation value calculating unit calculates a workflow step evaluation value of the workflow step item which constitutes the first workflow information associated with the first patient identifier, calculates a workflow evaluation value, which is an evaluation value on the basis of the evaluation of the individual medical-related works in the first workflow information, on the basis of the calculated workflow step evaluation value, and the screen output unit outputs the first workflow information and the workflow evaluation value to the screen.

7. The medical care support system according to claim 1, wherein
the medical information database stores reference information, which is information for identifying a workflow step referred to in the workflow step, in association with the workflow step information, and
the workflow step evaluation value calculating unit acquires the number of workflow steps that refers to the first workflow step item on the basis of the reference information and calculates the workflow step evaluation value on the basis of the acquired number of workflow steps.

8. The medical care support system according to claim 7, wherein
the medical information includes evidence data indicating biological information and data processing archival record information of the evidence data, and
the workflow step evaluation value calculating means acquires first data processing archival record information associated with the first workflow step item from the medical information database, acquires first reference information associated with the first workflow step item from the medical information database, acquires a reference data processing archival record, which is a data processing archival record in the workflow step referred to and identified by the first reference information, from the medical information database, calculates processing archival record coincidence between the first data processing archival record and the reference data processing archival record, and calculates the first workflow step evaluation value on the basis of the calculated processing archival record coincidence.

9. The medical care support system according to claim 8, wherein
the workflow step evaluation value calculating means calculates a coefficient of correlation between the calculated processing archival record coincidence and the workflow clinical evaluation value in the first workflow group, calculates a workflow step reproduction influence rate indicating the degree of improvement of a clinical indicator by processing archival record improvement on the basis of the calculated coefficient of correlation, and calculates the first workflow step evaluation value on the basis of the calculated workflow step reproduction influence rate, and
the screen output unit outputs the calculated workflow step reproduction influence rate and the calculated first workflow step evaluation value to the screen.
